(19) United States
(12) Patent Application Publication
(10) Pub. No.: US 2021/0401712 A1
(43) Pub. Date: Dec. 30, 2021

(54) METHOD FOR TREATING HAIR, COMPRISING THE APPLICATION OF AN ORGANIC SILICON COMPOUND, AN ALKALISING AGENT AND A FILM-FORMING POLYMER

(71) Applicant: Henkel AG & Co. KGaA, Duesseldorf (DE)

(72) Inventors: GABRIELE WESER, Neuss (DE); CLAUDIA KOLONKO, Remscheid (DE); ULRIKE SCHUMACHER, Duesseldorf (DE); CAROLINE KRIENER, Duesseldorf (DE); MARC NOWOTTNY, Moenchengladbach (DE); JUERGEN SCHOEPGENS, Schwalmtal (DE); CARSTEN MATHIASZYK, Essen (DE); TORSTEN LECHNER, Langenfeld (DE)

(73) Assignee: Henkel AG & Co. KGaA, Duesseldorf (DE)

(21) Appl. No.: 17/290,217

(22) PCT Filed: Aug. 12, 2019
(86) PCT No.: PCT/EP2019/071624

§ 371 Date: Apr. 29, 2021

(30) Foreign Application Priority Data
Oct. 31, 2018 (DE) 10 2018 218 636.7

(51) Int. Cl. A61K 8/58; A61K 8/19; A61K 8/73; A61K 8/81; A61K 8/891; A61Q 5/10; A61K 8/49; A61K 8/42

(52) U.S. Cl. CPC A61K 8/585; A61K 8/19; A61K 8/731; A61K 8/8147; A61K 8/891; A61K 2800/43; A61K 8/58; A61K 8/494; A61K 8/42; A61K 2800/5922; A61K 2800/432; A61Q 5/10

(57) ABSTRACT

It is an object of the present disclosure to provide a method for treating keratinous material, in particular human hair, comprising the following steps:
Application of a water-containing agent (a) to the keratinous material, wherein the agent (a) has and contains a pH of at least 9.6:
(a1) at least one organic silicon compound selected from the group including silanes having one, two or three silicon atoms, and
(a2) at least one alkalizing agent selected from the group including ammonia, alkanolamines and basic amino acids, and
Application of an agent (b) to the keratinous material, wherein the agent (b) includes:
(b1) at least one film-forming polymer.

### CROSS-REFERENCE TO RELATED APPLICATION

This application is a U.S. National-Stage entry under 35 U.S.C. § 371 based on International Application No. PCT/EP2019/071624, filed Aug. 12, 2019, which was published under PCT Article 21(2) and which claims priority to German Application No. 10 2018 218 636.7, filed Oct. 31, 2018, which are all hereby incorporated in their entirety by reference.

### TECHNICAL FIELD

The subject of the present application is a method for treating keratinous material, in particular human hair, which includes the application of two agents (a) and (b). The agent (a) contains water and has an alkaline pH value. Furthermore, the agent (a) is exemplified by its content of at least one organic silicon compound (a1) and at least one alkalizing agent (a2). The agent (b) contains at least one film-forming, polymer (b1).

A further subject of this application is a multi-component packaging unit (kit-of-parts) for dyeing keratinous material, in particular human hair, which includes separately prepared at least three means (a′), (a″) and (b). Agents (a′) and (a″) can be used to prepare the agent (a) used in the process described above.

### BACKGROUND

The change in shape and color of keratin fibers, especially hair, is an important area of modern cosmetics. To change the hair color, the expert knows various coloring systems depending on coloring requirements. Oxidation dyes are usually used for permanent, intensive dyeing's with good fastness properties and good grey coverage. Such dyes usually contain oxidation dye precursors, so-called developer components and coupler components, which form the actual dyes with one another under the influence of oxidizing agents, such as hydrogen peroxide. Oxidation dyes are exemplified by very long-lasting dyeing results.

When direct dyes are used, ready-made dyes diffuse from the colorant into the hair fiber. Compared to oxidative hair dyeing, the dyeing's obtained with direct dyes have a shorter shelf life and quicker wash ability. Dyeing with direct dyes usually remain on the hair for a period of between about 5 and about 20 washes.

The use of color pigments is known for short-term color changes on the hair and/or skin. Color pigments are generally understood to be insoluble, coloring substances. These are present undissolved in the dye formulation in the form of small particles and are only deposited from the outside on the hair fibers and/or the skin surface. Therefore, they can usually be removed again without residue by a few washes with detergents containing surfactants. Various products of this type are available on the market under the name hair mascara.

If the user wants particularly long-lasting dyeing's, the use of oxidative dyes has so far been his only option. However, despite numerous optimization attempts, an unpleasant ammonia or amine odor cannot be completely avoided in oxidative hair dyeing. The hair damage still associated with the use of oxidative dyes also has a negative effect on the user's hair.

EP 2168633 B1 deals with the task of producing long-lasting hair colorations using pigments. The paper teaches that when a combination of a pigment, an organic silicon compound, a film-forming polymer and a solvent is used on hair, it is possible to produce colorations that are particularly resistant to shampooing.

During the revision of the doctrine of EP 2168633 B1, its formulations have been adjusted. It has been shown that the disadvantages of these formulations are their still insufficient color intensities and inadequate wash fastness properties. Accordingly, the shampooing fastnesses of the colorations produced in EP 2168633 B1 still need improvement.

### BRIEF SUMMARY

The purpose of the present disclosure was to provide a dyeing system with fastness properties comparable to those of oxidative dyeing. In particular, the color intensities and wash fastnesses should be outstanding, but the use of the oxidation dye precursors normally used for this purpose should be avoided. A technology was sought that would make it possible to fix the coloring compounds known from state-of-the-art technology (such as pigments and direct dyes) to the hair in an extremely permanent manner Even after multiple washes (such as hair washes), the colorant compounds placed on the keratin materials should not detach from the keratin material.

Surprisingly, it has now been found that the task can be excellently solved if keratinous materials, in particular human hair, are treated or dyed by a process comprising the successive application of two agents (a) and (b). Here, the first agent (a) is an aqueous or water-containing agent with an alkaline pH of at least 9.6, which, in addition to at least one organic silicon compound, further contains at least one alkalizing agent. The second agent (b) includes at least one film-forming polymer.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

A first object of the present disclosure is a method for treating keratinous material, in particular human hair, comprising the following steps:
Application of a water-containing agent (a) to the keratinous material, wherein the agent (a) has and contains a pH of at least 9.6:
(a1) at least one organic silicon compound selected from the group including silanes having one, two or three silicon atoms, and
(a2) at least one alkalizing agent selected from the group including ammonia, alkanolamines and basic amino acids, and
Application of an agent (b) to the keratinous material, wherein the agent (b) includes:
(b1) at least one film-forming polymer.

In the work leading to the present invention, it has been found that the preferential successive application of agents (a) and (b) allows the formation of very stable films on the keratin materials. Without being limited to this theory, it is believed in this context that the joint application of organic silicon compound (a1) and alkalizing agent (a2) in the aqueous agent (a) initially led to the accelerated formation of an initial film on the keratin material. With application of the second agent (b), another polymer layer was now deposited on this first layer. Compared to an application without alkalizing agent, the multilayer film system produced in this way exhibited improved resistance to external influences. Colorant compounds, which can be incorporated into the agent (a) and/or into the agent (b), were permanently fixed to the keratin material in this way. In this way, extremely washfast colorations with good resistance to shampooing were obtained on the keratinous material.

### Process for Dyeing Keratinous Material

Keratinous material includes hair, skin, nails (such as fingernails and/or toenails). Wool, furs, and feathers also fall under the definition of keratinous material.

In an exemplary embodiment, keratinous material is understood to be human hair, human skin, and human nails, especially fingernails and toenails. Keratinous material is understood to be human hair.

### Agent (a) and (b)

In the procedure as contemplated herein, agents (a) and (b) are applied to the keratinous material, in particular human hair. The two means (a) and (b) are different.

In other words, a first object of the present disclosure is a method for treating keratinous material, in particular human hair, comprising the following steps:
Application of a water-containing agent (a) to the keratinous material, wherein the agent (a) has and contains a pH of at least 9.6:
(a1) at least one organic silicon compound selected from the group including silanes having one, two or three silicon atoms, and
(a2) at least one alkalizing agent selected from the group including ammonia, alkanolamines and basic amino acids, and
Application of an agent (b) to the keratinous material, wherein the agent (b) includes:
(b1) at least one film-forming polymer.
   where the two means (a) and (b) are different.

### Agent (a)

Agent (a) is aqueous, which means that agent (a) contains organic silicon compound(s) (a1) and alkalizing agent(s) (a2) in an aqueous or aqueous-alcoholic cosmetic carrier. This cosmetic carrier can be liquid, gel or cream. To hair treatment, in particular hair coloring, such carriers are, for example, creams, emulsions, gels, or also surfactant-containing foaming solutions, such as shampoos, foam aerosols, foam formulations or other preparations suitable for application to the hair.

In an exemplary embodiment, the cosmetic carrier contains—based on its weight—at least 2% by weight of water. Further in an exemplary embodiment, the water content is above 10% by weight, still further in an exemplary embodiment above 20% by weight and particularly above 40% by weight. The cosmetic carrier can also be aqueous-alcoholic. [0206] Aqueous/alcoholic solutions in the context of the invention are aqueous solutions containing about 2 to about 70% by weight of a C₁-C₄ alcohol, more particularly ethanol or isopropanol. The agents as contemplated herein may additionally contain other organic solvents, such as methoxy butanol, benzyl alcohol, ethyl diglycol or 1,2-propylene glycol. Suitable are all water-soluble organic solvents.

Another characteristic of agent (a) is its alkaline pH value of at least 9.6. For example, the pH value can be measured using a glass electrode, which is usually commercially available in the form of a combination electrode. Before measuring the pH value, the glass electrodes are usually calibrated with calibration solutions of known pH value. For the purposes of the present invention, pH values are understood to be pH values measured at a temperature of 22° C.

It has been shown that by adjusting the alkaline pH value to at least 9.6, particularly resistant films could be produced on the keratin materials. Particularly good results were obtained when the agent (a) was adjusted to a pH value of about 9.7 to about 11.5, in an exemplary embodiment about 9.8 to about 11.3, for example about 9.9 to about 11.0, and such as about 10.0 to about 10.9.

In a very particularly suitable embodiment, a process as contemplated herein is exemplified in that the agent (a) has a pH of from about 9.7 to about 11.5, in an exemplary embodiment from about 9.8 to about 11.3, more for example from about 9.9 to about 11.0 such as from about 10.0 to about 10.9.

Organic Silicon Compounds from the Group of Silanes (a1)

As an ingredient (a1) essential to the invention, the composition (a) contains at least one organic silicon compound from the group of silanes having one, two or three silicon atoms.

In an embodiment, the agent (a) contains at least one organic silicon compound (a1) selected from silanes having one, two or three silicon atoms, the organic silicon compound comprising one or more hydroxyl groups and/or hydrolyzable groups per molecule.

These organic silicon compounds (a1) or organic silanes contained in the agent (a) is reactive compounds.

Organic silicon compounds, alternatively called organosilicon compounds, are compounds which either have a direct silicon-carbon bond (Si—C) or in which the carbon is bonded to the silicon atom via an oxygen, nitrogen, or sulfur atom. The organic silicon compounds as contemplated herein are compounds containing one to three silicon atoms. Organic silicon compounds in an exemplary embodiment contain one or two silicon atoms.

According to IUPAC rules, the term silane chemical compounds based on a silicon skeleton and hydrogen. In organic silanes, the hydrogen atoms are completely or partially replaced by organic groups such as (substituted) alkyl groups and/or alkoxy groups. In organic silanes, some of the hydrogen atoms may also be replaced by hydroxy groups.

In a particularly suitable embodiment, a method as contemplated herein is exemplified by the application of an agent (a) to the keratinous material, said agent (a) comprising at least one organic silicon compound selected from silanes having one, two or three silicon atoms, said organic silicon compound further comprising one or more hydroxyl groups or hydrolyzable groups per molecule.

In a particularly suitable embodiment, a method as contemplated herein is exemplified by the application of an agent (a) to the keratinous material, said agent (a) comprising at least one organic silicon compound selected from silanes having one, two or three silicon atoms, said organic silicon compound further comprising one or more basic chemical functions and one or more hydroxyl groups or hydrolyzable groups per molecule.

This basic group can be, for example, an amino group, an alkylamino group or a dialkylamino group, which is in an exemplary embodiment connected to a silicon atom via a linker. The basic group is in an exemplary embodiment an amino group, a C₁-C₆ alkylamino group or a di(C₁-C₆)alkylamino group.

The hydrolyzable group(s) is (are) in an exemplary embodiment a C₁-C₆ alkoxy group, especially an ethoxy group or a methoxy group. It is suitable when the hydrolyzable group is directly bonded to the silicon atom. For example, if the hydrolyzable group is an ethoxy group, the organic silicon compound in an exemplary embodiment contains a structural unit R′R″R′″Si—O—CH2-CH3. The residues R, R′ and R″ represent the three remaining free valences of the silicon atom.

A particularly suitable method as contemplated herein is exemplified in that the composition includes (a) at least one organic silicon compound selected from silanes having one, two or three silicon atoms, the organic silicon compound in an exemplary embodiment comprising one or more basic chemical functions and one or more hydroxyl groups or hydrolyzable groups per molecule.

Very particularly good results could be obtained if the agent (a) as contemplated herein contains at least one organic silicon compound (a1) of formula (I) and/or (II).

The compounds of formulae (I) and (II) are organic silicon compounds selected from silanes having one, two or three silicon atoms, the organic silicon compound comprising one or more hydroxyl groups and/or hydrolysable groups per molecule.

In another particularly suitable embodiment, a method as contemplated herein is exemplified in that an agent (a) is applied to the keratinous material (or human hair), the agent (a) containing at least one organic silicon compound of formula (I) and/or (II).

R₁R₂N-L-Si(OR₃)ₐ(R₄)_{b} (I),

where
R₁, R₂ independently represent a hydrogen atom or a C₁-C₆ alkyl group,
L is a linear or branched divalent C₁-C₂₀ alkylene group,
R₃ represents a hydrogen atom or a C₁-C₆ alkyl group,
R₄ represents a C₁-C₆ alkyl group
a, stands for an integer from 1 to 3, and
b stands for the integer 3-a,

(R₅O)_{c}(R₆)_{d}Si-(A)ₑ-[NR₇-(A′)]_{f}—[O-(A″)]_{g}—[NR₈-(A′″)]ₕ—Si(R₆′)_{d′}(OR₅′)_{c′} (II),

where
R5, R5′, R5″ independently represent a hydrogen atom or a C₁-C₆ alkyl group,
R6, R6′ and R6″ independently represent a C₁-C₆ alkyl group,
A, A′, A″, independently of one another represent a linear or branched divalent C₁-C₂₀ alkylene group
R₇ and R₈ independently represent a hydrogen atom, a C₁-C₆ alkyl group, a hydroxy C₁-C₆ alkyl group, a C₂-C₆ alkenyl group, an amino C₁-C₆ alkyl group or a group of formula (III)

-(A″″)-Si(R₆″)_{d}″(OR₅″)_{c}″ (III),
c, stands for an integer from 1 to 3,
d stands for the integer 3-c,
c′ stands for an integer from 1 to 3,
d′ stands for the integer 3-c′,
c″ stands for an integer from 1 to 3,
d″ stands for the integer 3-c″,
e stands for 0 or 1,
f stands for 0 or 1,
g stands for 0 or 1,
h stands for 0 or 1,
provided that at least one of e, f, g, and h is different from 0.

The substituents R₁, R₂, R₃, R₄, R₅, R₅′, R₅″, R₆, R₆′, R₆″, R₇, R₈, L, A′, A″″ and A″″ in the compounds of formula (I) and (II) are explained below as examples:

Examples of a C₁-C₆ alkyl group are the groups methyl, ethyl, propyl, isopropyl, n-butyl, s-butyl, and t-butyl, n-pentyl and n-hexyl. Propyl, ethyl, and methyl are suitable alkyl radicals. Examples of a C₂-C₆ alkenyl group are vinyl, allyl, but-2-enyl, but-3-enyl and isobutenyl, suitable C₂-C₆ alkenyl radicals are vinyl and allyl. Suitable examples of a hydroxy C₁-C₆ alkyl group are a hydroxymethyl, a 2-hydroxyethyl, a 2-hydroxypropyl, a 3-hydroxypropyl, a 4-hydroxybutyl group, a 5-hydroxypentyl and a 6-hydroxyhexyl group; a 2-hydroxyethyl group is particularly suitable. Examples of an amino C₁-C₆ alkyl group are the aminomethyl group, the 2-aminoethyl group, the 3-aminopropyl group. The 2-aminoethyl group is particularly suitable. Examples of a linear divalent C₁-C₂₀ alkylene group include the methylene group (—CH₂), the ethylene group (—CH₂—CH₂—), the propylene group (—CH₂—CH₂—CH₂—) and the butylene group (—CH₂—CH₂—CH₂—). The propylene group (—CH₂—CH₂—CH₂—) is particularly suitable. From a chain length of 3 C atoms, divalent alkylene groups can also be branched. Examples of branched divalent C₃-C₂₀ alkylene groups are (—CH₂—CH(CH₃)—) and (—CH₂—CH(CH₃)—CH₂—).

In the organic silicon compounds of the formula (I)

R₁R₂N-L-Si(OR₃)ₐ(R₄)_{b} (I),

the radicals R₁ and R₂ independently of one another represent a hydrogen atom or a C₁-C₆ alkyl group. In particular, the radicals R₁ and R₂ both represent a hydrogen atom.

In the middle part of the organic silicon compound is the structural unit or the linker -L- which stands for a linear or branched, divalent C₁-C₂₀ alkylene group.

A divalent C₁-C₂₀ alkylene group may alternatively be referred to as a divalent or divalent C₁-C₂₀ alkylene group, by which is meant that each L grouping may form two bonds. One bond is from the amino group R1R2N to the linker L, and the second bond is between the linker L and the silicon atom.

In an exemplary embodiment, -L- represents a linear, divalent (i.e., divalent) C₁-C₂₀ alkylene group. Further in an exemplary embodiment -L- stands for a linear divalent C₁-C₆ alkylene group. Particularly suitable -L stands for a methylene group (CH₂—), an ethylene group (—CH₂—CH₂—), propylene group (—CH₂—CH₂—CH₂—) or butylene (—CH₂—CH₂—CH₂—CH₂—). L stands for a propylene group (—CH₂—CH₂—CH₂—)

The linear propylene group (—CH₂—CH₂—CH₂—) can alternatively be referred to as the propane-1,3-diyl group.

The organic silicon compounds of formula (I)

R₁R₂N-L-Si(OR₃)ₐ(R₄)_{b}

one end of each carries the silicon-containing group —Si(OR₃)ₐ(R₄)_{b}

In the terminal structural unit —Si(OR₃)ₐ(R₄)_{b}, R₃ is hydrogen or C₁-C₆ alkyl group, and R₄ is C₁-C₆ alkyl group. R3 and R₃ independently of each other represent a methyl group or an ethyl group.

Here a stands for an integer from 1 to 3, and b stands for the integer 3-a. If a stands for the number 3, then b is equal to 0. If a stands for the number 2, then b is equal to 1. If a stands for the number 1, then b is equal to 2.

Particularly resistant films could be produced if the agent (a) contains at least one organic silicon compound (a1) of formula (I) in which the radicals R₃, R₄ independently of one another represent a methyl group or an ethyl group.

When using the process as contemplated herein for dyeing keratin material, dyeing's with the best wash fastnesses could be obtained analogously when the agent (a) contains at least one organic silicon compound of formula (I) in which the radicals R₃, R₄ independently of one another represent a methyl group or an ethyl group.

Furthermore, dyeing's with the best wash fastness properties could be obtained if the agent as contemplated herein contains at least one organic silicon compound of formula (I) in which the radical a represents the number 3. In this case the rest b stands for the number 0.

In a further suitable embodiment, the agent (a) used in the process as contemplated herein is exemplified in that it includes at least one organic silicon compound (a1) of the formula (I), where
R₃, R₄ independently of one another represent a methyl group or an ethyl group and
a stands for the number 3 and
b stands for the number 0.

In a further suitable embodiment, a process as contemplated herein is exemplified in that the agent (a) includes at least one organic silicon compound (a1) of the formula (I),

R₁R₂N-L-Si(OR₃)ₐ(R₄)_{b} (I),

where
R₁, R₂ both represent a hydrogen atom, and
L represents a linear, divalent C₁-C₆-alkylene group, in an exemplary embodiment a propylene group (—CH₂—CH₂—CH₂—) or an ethylene group (—CH₂—CH₂—),
R₃ represents a hydrogen atom, an ethyl group, or a methyl group,
R₄ represents a methyl group or an ethyl group,
a stands for the number 3 and
b stands for the number 0.

Organic silicon compounds of the formula (I) which are particularly suitable for solving the problem as contemplated herein are

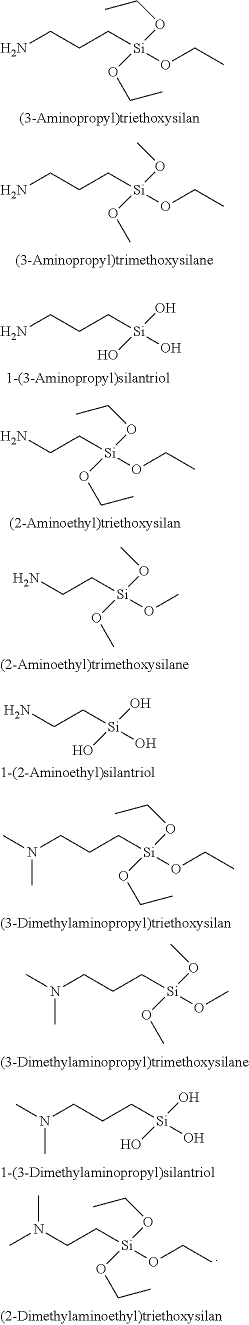

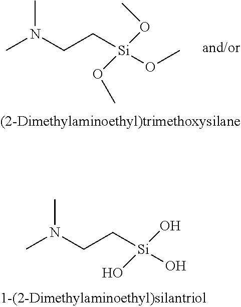

In a further suitable embodiment, a process as contemplated herein is exemplified in that the agent (a) includes at least one organic silicon compound (a1) selected from the group of
(3-Aminopropyl)triethoxysilan
(3-Aminopropyl)trimethoxysilane
1-(3-Aminopropyl)silantriol
(2-Aminoethyl)triethoxysilan
(2-Aminoethyl)trimethoxysilane
1-(2-Aminoethyl)silantriol
(3-Dimethylaminopropyl)triethoxysilan
(3-Dimethylaminopropyl)trimethoxysilane
1-(3-Dimethylaminopropyl)silantriol
(2-Dimethylaminoethyl)triethoxysilan.
(2-Dimethylaminoethyl)trimethoxysilane and/or
1-(2-Dimethylaminoethyl)silantriol.

The organic silicon compounds of formula (I) are commercially available. (3-aminopropyl)trimethoxysilane, for example, can be purchased from Sigma-Aldrich. Also (3-aminopropyl)triethoxysilane is commercially available from Sigma-Aldrich.

In a further embodiment, the composition as contemplated herein includes at least one organic silicon compound (a1) of the formula (II)

(R₅O)_{c}(R₆)_{d}Si-(A)ₑ-[NR₇-(A′)]_{f}—[O-(A″)]_{g}—[NR₈-(A′″)]ₕ—Si(R₆′)_{d′}(OR₅′)_{c′} (II).

The organosilicon compounds of formula (II) as contemplated herein each carry the silicon-containing groups (R₅O)_{c}(R₆)_{d}Si— and —Si(R₆′)_{d′}(OR₅′)_{c} at both ends.

In the central part of the molecule of formula (II) there are the groups -(A)ₑ- and —[NR₇-(A′)]_{f}- and —[O-(A″)]_{g}- and —[NR₈-(A′″)]ₕ-. Here, each of the radicals e, f, g, and h can independently of one another stand for the number 0 or 1, with the proviso that at least one of the radicals e, f, g, and h is different from 0. In other words, an organic silicon compound of formula (II) as contemplated herein contains at least one grouping from the group including -(A)- and —[NR₇-(A′)]- and —[O-(A″)]- and —[NR₈-(A′″)]-.

In the two terminal structural units (R₅O)_{c}(R₆)_{d}Si— and —Si(R₆′)_{d′}(OR₅′)_{c}, the radicals R5, R5′, R5″ independently of one another represent a hydrogen atom or a C₁-C₆ alkyl group. The radicals R6, R6′ and R6″ independently represent a C₁-C₆ alkyl group.

Here a stands for an integer from about 1 to about 3, and d stands for the integer 3-c. If c stands for the number 3, then d is equal to 0. If c stands for the number 2, then d is equal to 1. If c stands for the number 1, then d is equal to 2.

Analogously c′ stands for a whole number from about 1 to about 3, and d′ stands for the whole number 3-c′. If c′ stands for the number 3, then d′ is 0. If c′ stands for the number 2, then d′ is 1. If c′ stands for the number 1, then d′ is 2.

Films with the highest stability or dyes with the best wash fastnesses could be obtained when the residues c and c′ both stand for the number 3. In this case d and d′ both stand for the number 0.

In a further suitable embodiment, a process as contemplated herein is exemplified in that the agent (a) includes at least one organic silicon compound (a1) of the formula (II),

(R₅O)_{c}(R₆)_{d}Si-(A)ₑ-[NR₇-(A′)]_{f}—[O-(A″)]_{g}—[NR₈-(A′″)]ₕ—Si(R₆′)_{d′}(OR₅′)_{c′} (II),

where
R5 and R5′ independently represent a methyl group or an ethyl group,
c and c′ both stand for the number 3 and
d and d′ both stand for the number 0.

If c and c′ are both the number 3 and d and d′ are both the number 0, the organic silicon compound of the invention corresponds to formula (Ha)

(R₅O)₃Si-(A)ₑ-[NR₇-(A′)]_{f}—[O-(A″)]_{g}—[NR₈-(A′″)]ₕ—Si(OR₅′)₃ (IIa).

The radicals e, f, g, and h can independently stand for the number 0 or 1, whereby at least one radical from e, f, g, and h is different from zero. The abbreviations e, f, g, and h thus define which of the groupings -(A)e- and -[NR7-(A′)]f- and —[O-(A″)]g- and -[NR8-(A′″)]h- are in the middle part of the organic silicon compound of formula (II).

In this context, the presence of certain groupings has proved to be particularly beneficial in terms of increasing washability. Particularly good results were obtained when at least two of the residues e, f, g, and h stand for the number 1. Especially suitable e and f both stand for the number 1. Furthermore, g and h both stand for the number 0.

If e and f both stand for the number 1 and g and h both stand for the number 0, the organic silicon compound as contemplated herein corresponds to formula (IIb)

(R₅O)_{c}(R₆)_{d}Si-(A)-[NR₇-(A′)]—Si(R₆′)_{d′}(OR₅′)_{c′} (IIb).

The radicals A, A′, A″, A′″ and A″″ independently represent a linear or branched divalent C₁-C₂₀ alkylene group. In an exemplary embodiment the radicals A, A′, A″, A′″ and A″″ independently of one another represent a linear, divalent C₁-C₂₀ alkylene group. Further in an exemplary embodiment the radicals A, A′, A″, A′″ and A″″ independently represent a linear divalent C₁-C₆ alkylene group. In particular, the radicals A, A′, A″, A′″ and A″″ independently of one another represent a methylene group (—CH₂—), an ethylene group (—CH₂—CH₂—), a propylene group (—CH₂—CH₂—CH₂—) or a butylene group (—CH₂—CH₂—CH₂—CH₂—). In particular, the residues A, A′, A″, A′″ and A″″ stand for a propylene group (—CH₂—CH₂—CH₂—).

The divalent C₁-C₂₀ alkylene group may alternatively be referred to as a divalent or divalent C₁-C₂₀ alkylene group, by which is meant that each grouping A, A′, A″, A′″ and A″″ may form two bonds.

The linear propylene group (—CH₂—CH₂—CH₂—) can alternatively be referred to as the propane-1,3-diyl group.

If the radical f represents the number 1, then the organic silicon compound of formula (II) as contemplated herein contains a structural grouping —[NR₇-(A′)]-.

If the radical f represents the number 1, then the organic silicon compound of formula (II) as contemplated herein contains a structural grouping —[NR₈-(A′″)]-.

Wherein R₇ and R₇ independently represent a hydrogen atom, a C₁-C₆ alkyl group, a hydroxy-C₁-C₆ alkyl group, a C₂-C₆ alkenyl group, an amino-C₁-C₆ alkyl group or a group of the formula (III)

-(A″″)-Si(R₆″)_{d}″(OR₅″)_{c}″ (III).

in an exemplary embodiment the radicals R7 and R8 independently of one another represent a hydrogen atom, a methyl group, a 2-hydroxyethyl group, a 2-alkenyl group, a 2-aminoethyl group or a grouping of the formula (III).

If the radical f represents the number 1 and the radical h represents the number 0, the organic silicon compound as contemplated herein contains the grouping [NR₇-(A′)] but not the grouping —[NR₈-(A′″)]. If the radical R7 now stands for a grouping of the formula (III), the agent (a) contains an organic silicone compound with 3 reactive silane groups.

In a further suitable embodiment, a process as contemplated herein is exemplified in that the agent (a) includes at least one organic silicon compound (a1) of the formula (II),

(R₅O)_{c}(R₆)_{d}Si-(A)ₑ-[NR₇-(A′)]_{f}—[O-(A″)]_{g}—[NR₈-(A′″)]ₕ—Si(R₆′)_{d′}(OR₅′)_{c′} (II),

where
e and f both stand for the number 1,
g and h both stand for the number 0,
A and A′ independently represent a linear, divalent C₁-C₆ alkylene group
   and
R₇ represents a hydrogen atom, a methyl group, a 2-hydroxyethyl group, a 2-alkenyl group, a 2-aminoethyl group or a group of formula (III).

In a further suitable embodiment, a process as contemplated herein is exemplified in that the agent (a) contains at least one organic silicon compound of the formula (II), where
e and f both stand for the number 1,
g and h both stand for the number 0,
A and A′ independently of one another represent a methylene group (—CH₂—), an ethylene group (—CH₂—CH₂—) or a propylene group (—CH₂—CH₂—CH₂),
   and
R₇ represents a hydrogen atom, a methyl group, a 2-hydroxyethyl group, a 2-alkenyl group, a 2-aminoethyl group or a group of formula (III).

Organic silicon compounds of the formula (II) which are well suited for solving the problem as contemplated herein are

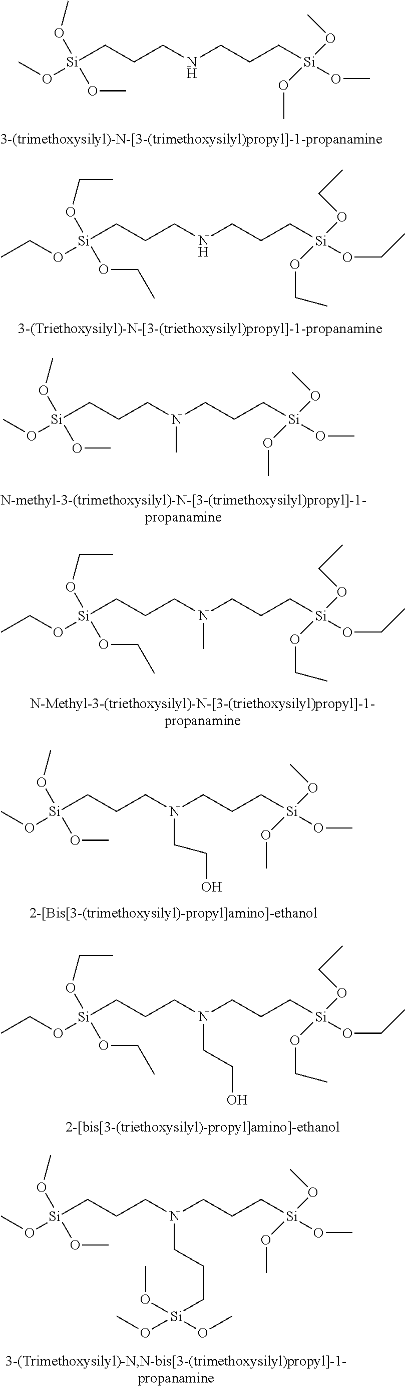

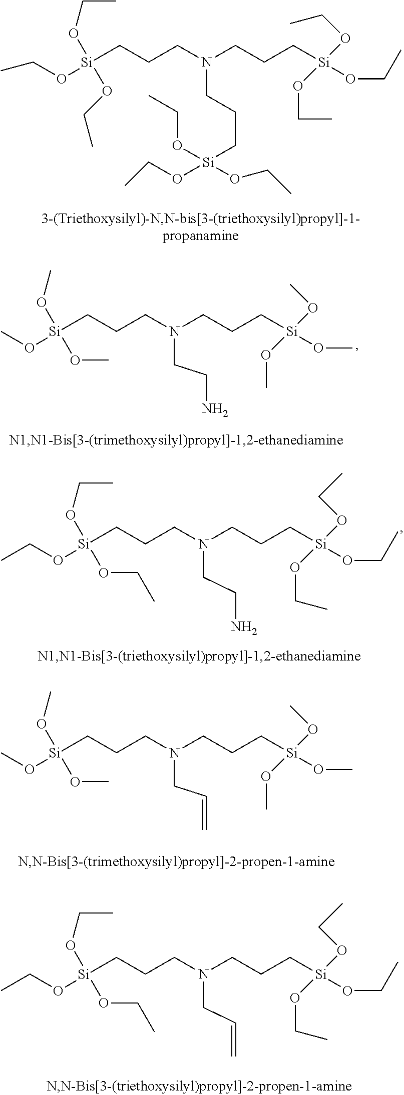

The organic silicon compounds of formula (II) are commercially available.
Bis(trimethoxysilylpropyl)amines with the CAS number 82985-35-1 can be purchased from Sigma-Aldrich.
Bis[3-(triethoxysilyl)propyl]amines with the CAS number 13497-18-2 can be purchased from Sigma-Aldrich, for example.
N-methyl-3-(trimethoxysilyl)-N-[3-(trimethoxysilyl)propyl]-1-propanamine is alternatively referred to as bis(3-trimethoxysilylpropyl)-N-methylamine and can be purchased commercially from Sigma-Aldrich or Fluorochem.
3-(triethoxysilyl)-N,N-bis[3-(triethoxysilyl)propyl]-1-propanamine with the CAS number 18784-74-2 can be purchased for example from Fluorochem or Sigma-Aldrich.

In a further suitable embodiment, a process as contemplated herein is exemplified in that the agent (a) includes at least one organic silicon compound (a1) selected from the group of
3-(trimethoxysilyl)-N-[3-(trimethoxysilyl)propyl]-1-propanamine
3-(Triethoxysilyl)-N-[3-(triethoxysilyl)propyl]-1-propanamine
N-methyl-3-(trimethoxysilyl)-N-[3-(trimethoxysilyl)propyl]-1-propanamine
N-Methyl-3-(triethoxysilyl)-N-[3-(triethoxysilyl)propyl]-1-propanamine
2-[Bis[3-(trimethoxysilyl)propyl]amino]-ethanol
2-[bis[3-(triethoxysilyl)propyl]amino]ethanol
3-(Trimethoxysilyl)-N,N-bis[3-(trimethoxysilyl)propyl]-1-propanamine
3-(Triethoxysilyl)-N,N-bis[3-(triethoxysilyl)propyl]-1-propanamine
N1,N1-bis[3-(trimethoxysilyl)propyl]-1,2-ethanediamine,
N1,N1-bis[3-(triethoxysilyl)propyl]-1,2-ethanediamine,
N,N-bis[3-(trimethoxysilyl)propyl]-2-propen-1-amine and/or
N,N-bis[3-(triethoxysilyl)propyl]-2-propen-1-amine

In further tests, in particular dyeing tests, it has also been found to be particularly advantageous if the agent (a) applied to the keratinous material in the process as contemplated herein contains at least one organic silicon compound of the formula (IV)

R₉Si(OR₁₀)ₖ(R₁₁)ₘ (IV).

The compounds of formula (IV) are organic silicon compounds selected from silanes having one, two or three silicon atoms, the organic silicon compound comprising one or more hydroxyl groups and/or hydrolysable groups per molecule.

The organic silicon compound(s) of formula (IV) may also be called a silane of the alkyl-alkoxy-silane or alkyl-hydroxy-silane type,

R₉Si(OR₁₀)ₖ(R₁₁)ₘ (IV),

where
R₉ represents a C₁-C₁₂ alkyl group,
R₁₀ represents a hydrogen atom or a C₁-C₆ alkyl group,
R₁₁ represents a C₁-C₆ alkyl group
k is an integer from 1 to 3, and
m stands for the integer 3-k.

In a further suitable embodiment, a process as contemplated herein is exemplified in that the agent (a) includes at least one organic silicon compound (a1) of the formula (IV)

R₉Si(OR₁₀)ₖ(R₁₁)ₘ (IV),

where
R₉ represents a C₁-C₁₂ alkyl group,
R₁₀ represents a hydrogen atom or a C₁-C₆ alkyl group,
R₁₁ represents a C₁-C₆ alkyl group
k is an integer from 1 to 3, and
m stands for the integer 3-k.

In a further suitable embodiment, a process as contemplated herein is exemplified in that the agent (a) contains, in addition to the organic silicon compound(s) of formula (I), at least one further organic silicon compound of formula (IV)

R₉Si(OR₁₀)ₖ(R₁₁)ₘ (IV),

where
R₉ represents a C₁-C₁₂ alkyl group,
R₁₀ represents a hydrogen atom or a C₁-C₆ alkyl group,
R₁₁ represents a C₁-C₆ alkyl group
k is an integer from 1 to 3, and
m stands for the integer 3-k.

In a further suitable embodiment, a process as contemplated herein is exemplified in that the agent (a) contains, in addition to the organic silicon compound(s) of formula (II), at least one further organic silicon compound of formula (IV)

R₉Si(OR₁₀)ₖ(R₁₁)ₘ (IV),

where
R₉ represents a C₁-C₁₂ alkyl group,
R₁₀ represents a hydrogen atom or a C₁-C₆ alkyl group,
R₁₁ represents a C₁-C₆ alkyl group
k is an integer from 1 to 3, and
m stands for the integer 3-k.

In a further suitable embodiment, a process as contemplated herein is exemplified in that the composition contains (a) in addition to the organic silicon compound(s) of formula (I) and/or (II) at least one further organic silicon compound of formula (IV)

R₉Si(OR₁₀)ₖ(R₁₁)ₘ (IV),

where
R₉ represents a C₁-C₁₂ alkyl group,
R₁₀ represents a hydrogen atom or a C₁-C₆ alkyl group,
R₁₁ represents a C₁-C₆ alkyl group
k is an integer from 1 to 3, and
m stands for the integer 3-k.

In the organic silicon compounds of formula (IV), the radical R₉ represents a C₁-C₁₂ alkyl group. This C₁-C₁₂ alkyl group is saturated and can be linear or branched. In an exemplary embodiment, R₉ represents a linear C₁-C₈ alkyl group. In an exemplary embodiment R₉ stands for a methyl group, an ethyl group, an n-propyl group, an n-butyl group, an n-pentyl group, an n-hexyl group, an n-octyl group or an n-dodecyl group. In an exemplary embodiment, R₉ represents a methyl group, an ethyl group or an n-octyl group.

In the organic silicon compounds of formula (IV), the radical R10 represents a hydrogen atom or a C₁-C₆ alkyl group. R10 stands for a methyl group or an ethyl group.

In the organic silicon compounds of formula (IV), the radical R₁₁ represents a C₁-C₆ alkyl group. R11 stands for a methyl group or an ethyl group.

Furthermore, k stands for a whole number from 1 to 3, and m stands for the whole number 3-k. If k stands for the number 3, then m is equal to 0. If k stands for the number 2, then m is equal to 1. If k stands for the number 1, then m is equal to 2.

Particularly stable films, i.e., dyeing's with particularly good wash fastness properties, could be obtained if an agent (a) containing at least one organic silicon compound corresponding to formula (IV): in which the radical k is the number 3, was used in the process. In this case the rest m stands for the number 0.

Organic silicon compounds of the formula (IV) which are particularly suitable for solving the problem as contemplated herein are

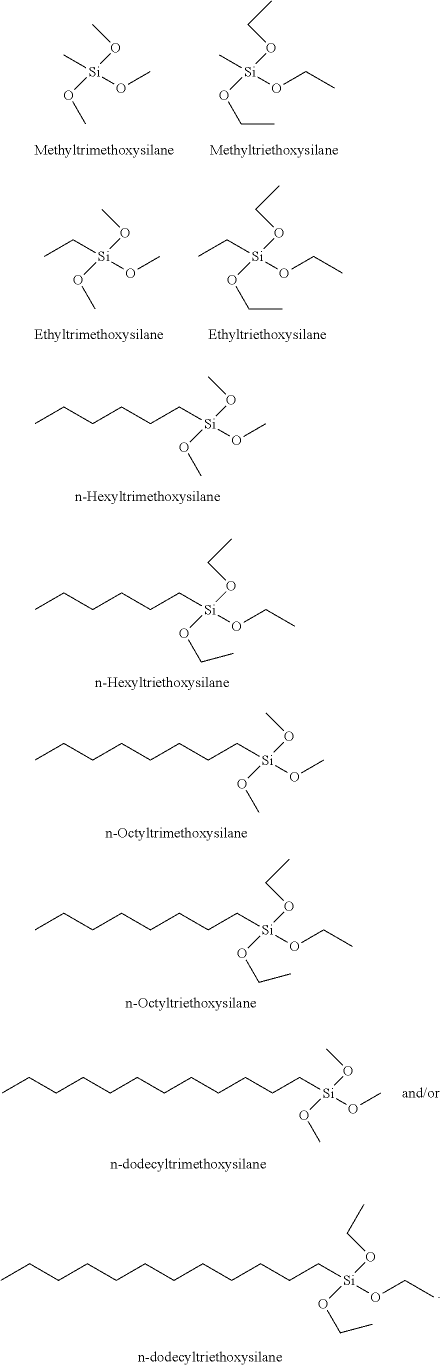

In a further suitable embodiment, a process as contemplated herein is exemplified in that the agent (a) includes at least one organic silicon compound (a1) of formula (IV) selected from the group including
Methyltrimethoxysilane
Methyltriethoxysilane
Ethyltrimethoxysilane
Ethyltriethoxysilane
Hexyltrimethoxysilane
Hexyltriethoxysilane
Octyltrimethoxysilane
Octyltriethoxysilane
Dodecyltrimethoxysilane and/or
Dodecyltriethoxysilane.

The organic silicon compounds described above are reactive compounds. In this context, it has been found preferable if the agent (a) as contemplated herein contains—based on the total weight of the agent (a)—one or more organic silicon compounds (a1) in a total amount of about 0.1 to about 20.0% by weight, for example about 1.0 to about 15.0% by weight and in an exemplary embodiment about 2.0 to about 8.0% by weight.

In a further suitable embodiment, a process as contemplated herein is exemplified in that the agent (a) contains—based on the total weight of the agent (a)—one or more organic silicon compounds (a1) in a total amount of about 0.1 to about 20.0% by weight, in an exemplary embodiment about 1.0 to about 15.0% by weight and in an exemplary embodiment about 2.0 to about 8.0% by weight.

To achieve particularly good dyeing results, it is particularly advantageous to use the organic silicon compounds of the formula (I) and/or (II) in certain quantity ranges on average (a). In an exemplary embodiment, the agent (a) contains—based on the total weight of the agent (a)—one or more organic silicon compounds of the formula (I) and/or (II) in a total amount of about 0.1 to about 10.0% by weight, in an exemplary embodiment about 0.5 to about 5.0% by weight and in an exemplary embodiment about 0.5 to about 3.0% by weight.

In a further suitable embodiment, a process as contemplated herein is exemplified in that the agent (a)—based on the total weight of agent (a)—contains one or more organic silicon compounds of formula (I) and/or (II) in a total amount of about 0.1 to about 10.0% by weight, in an exemplary embodiment about 0.5 to about 5.0% by weight and in an exemplary embodiment about 0.5 to about 3.0% by weight.

Furthermore, it has proven to be particularly suitable if the organic silicon compound(s) of formula (IV) is (are) also present in certain quantity ranges in average (a). In an exemplary embodiment the agent (a) contains—based on the total weight of agent (a)—one or more organic silicon compounds of the formula (IV) in a total amount of about 0.1 to about 20.0% by weight, in an exemplary embodiment about 2.0 to about 15.0% by weight and in an exemplary embodiment about 4.0 to about 9.0% by weight.

In a further suitable embodiment, a process as contemplated herein is exemplified in that the agent (a)—based on the total weight of agent (a)—contains one or more organic silicon compounds of formula (IV) in a total amount of about 0.1 to about 20.0% by weight, in an exemplary embodiment about 2.0 to about 15.0% by weight and in an exemplary embodiment about 3.2 to about 10.0% by weight.

In the course of the work leading to this invention it turned out that particularly stable and uniform films could be obtained on the keratin material if the agent (a) contains two structurally different organic silicon compounds.

In another suitable embodiment, a process as contemplated herein is exemplified in that the agent (a) contains at least two structurally different organic silicon compounds.

In an explicitly particularly suitable embodiment, a process as contemplated herein is exemplified in that an agent (a) is applied to the keratinous material which contains at least one organic silicon compound of the formula (I) which is selected from the group including (3-aminopropyl)triethoxysilane and (3-aminopropyl)trimethoxysilane, and additionally contains at least one organic silicon compound of the formula (IV) which is selected from the group including methyltrimethoxysilane, methyltriethoxysilane, ethyltrimethoxysilane and ethyltriethoxysilane.

In a further suitable embodiment, a process as contemplated herein is exemplified in that the agent (a)—based on the total weight of agent (a)—contains:
0.5 to 5.0 weight % of at least one first organic silicon compound (a1) selected from the group of (3-aminopropyl)trimethoxysilane, (3-aminopropyl)triethoxysilane, (2-aminoethyl)trimethoxysilane, (2-aminoethyl)triethoxysilane, (3-dimethylaminopropyl)trimethoxysilane, (3-dimethylaminopropyl)triethoxysilane (2-dimethylaminoethyl)trimethoxysilane and (2-dimethylaminoethyl)triethoxysilane, and
3.2 to 10.0% by weight of at least one second organic silicon compound (a1) selected from the group of methyltrimethoxysilane, methyltriethoxysilane, ethyltrimethoxysilane, ethyltriethoxysilane, octyltrimethoxysilane, octyltriethoxysilane, dodecyltrimethoxysilane and dodecyltriethoxysilane.

In this version, the agent contains (a) one or more organic silicon compounds of a first group in a total amount of about 0.5 to about 3.0% by weight. The organic silicon compounds of this first group are selected from the group of (3-aminopropyl)trimethoxysilane, (3-aminopropyl)triethoxysilane, (2-aminoethyl)trimethoxysilane, (2-aminoethyl)triethoxysilane, (3-dimethylaminopropyl)trimethoxysilane, (3-dimethylaminopropyl)triethoxysilane (2-dimethylaminoethyl)trimethoxysilane and/or (2-dimethylaminoethyl)triethoxysilane.

In this version, the agent contains (a) one or more organic silicon compounds of a second group in a total amount of about 3.2 to about 10.0% by weight. The organic silicon compounds of this second group are selected from the group of methyltrimethoxysilane, methyltriethoxysilane, ethyltrimethoxysilane, ethyltriethoxysilane, octyltrimethoxysilane, octyltriethoxysilane, dodecyltrimethoxysilane and/or dodecyltriethoxysilane.

### Alkalizing Agent (a2)

As an ingredient (a2) essential to the invention, the composition (a) contains at least one alkalizing agent selected from the group including ammonia, alkanolamines and basic amino acids.

Ammonia was found to exert a particularly strong influence on the optimization of the oligomerization or polymerization reaction of the organic silicon compounds (a1). When ammonia was used, it was therefore possible to produce particularly resistant films. Accordingly, dyeing's with the highest color intensity and the best wash fastnesses could also be obtained when the agent (a) contained ammonia as alkalizing agent (a2).

In an explicitly quite particularly suitable embodiment, a process as contemplated herein is exemplified in that the agent (a) contains ammonia as alkalizing agent (a2).

Good results were also obtained when the agent (a) contained at least one alkanolamine as alkalizing agent (a2).

The alkanolamines that can be used in agent (a) can be selected, for example, from the group of primary amines having a C2-C6 alkyl parent carrying at least one hydroxyl group. Suitable alkanolamines are selected from the group formed by 2-aminoethan-1-ol (monoethanolamine), 3-aminopropan-1-ol, 4-aminobutan-1-ol, 5-aminopentan- 1-ol, 1-aminopropan-2-ol, 1-aminobutan-2-ol, 1-aminopentan-2-ol, 1-aminopentan-3-ol, 1-aminopentan-4-ol, 3-amino-2-methylpropan-1-ol, 1-amino-2-methylpropan-2-ol, 3-aminopropan-1,2-diol, 2-amino-2-methylpropan-1,3-diol.

In a further suitable embodiment, a process as contemplated herein is exemplified in that the agent (a) includes at least one alkalizing agent (a2) from the group including the alkanolamines, which is in an exemplary embodiment selected from the group including 2-aminoethan-1-ol (monoethanolamine), 3-aminopropan-1-ol, 4-aminobutan- 1-ol, 5-aminopentan-1-ol, 1-aminopropan-2-ol, 1-aminobutan-2-ol, 1-aminopentan-2-ol, 1-aminopentan-3-ol, 1-aminopentan-4-ol, 3-amino-2-methylpropan-1-ol, 1-amino-2-methylpropan-2-ol, 3-aminopropane-1,2-diol and 2-amino-2-methylpropane-1,3-diol.

Good results were also obtained when the agent (a) contained at least one basic amino acid as alkalizing agent (a2).

For the purposes of the invention, an amino acid is an organic compound containing in its structure at least one protonatable amino group and at least one —COOH or one —SO₃H group. Suitable amino acids are aminocarboxylic acids, especially α-(alpha)-aminocarboxylic acids and ω-aminocarboxylic acids, whereby α-aminocarboxylic acids are particularly suitable.

As contemplated herein, basic amino acids are those amino acids which have an isoelectric point pI of greater than 7.0.

Basic α-aminocarboxylic acids contain at least one asymmetric carbon atom. In the context of the present invention, both possible enantiomers can be used equally as specific compounds or their mixtures, especially as racemates. However, it is particularly advantageous to use the naturally suitable isomeric form, usually in L-configuration.

The basic amino acids are in an exemplary embodiment selected from the group formed by arginine, lysine, ornithine, and histidine, especially in an exemplary embodiment arginine and lysine. In another particularly suitable embodiment, an agent as contemplated herein is therefore exemplified in that the alkalizing agent is a basic amino acid from the group arginine, lysine, ornithine and/or histidine.

In a further suitable embodiment, a process as contemplated herein is exemplified in that the agent (a) contains at least one alkalizing agent (a2) from the group of basic amino acids, which is in an exemplary embodiment selected from the group including arginine, lysine, ornithine, and histidine.

Suitable agents (a) as contemplated herein may also contain alkalizing agent mixtures, such as a mixture of ammonia and alkanolamine or a mixture of ammonia and basic amino acid.

Particularly suitable are the alkalizing agents used in certain combinations: Ammoniak/2-Aminoethan-1-ol; Ammoniak/2-Amino-2-methylpropan-1-ol; Ammoniak/Arginin; Ammoniak/Lysin; Ammoniak/Ornithin and/or Ammoniak/Histidin;

Although the agents (a) as contemplated herein are adjusted to pH values in the alkaline range, it may nevertheless be necessary in principle to also use acidifiers in small amounts for fine adjustment of the desired pH value. Acidifiers suitable as contemplated herein are, for example, citric acid, lactic acid, acetic acid or also dilute mineral acids (such as hydrochloric acid, sulfuric acid, phosphoric acid).

However, in the course of the work leading to the present invention, it has been found that the presence of the alkalizing agent or the adjustment of the alkaline pH is essential for the formation of resistant films on the keratin material. The presence of excessive amounts of acids can have a negative effect on the strength of the films. For this reason, it has proved preferable to keep the quantities of acids used in the medium (a) as low as possible. For this reason, it is advantageous if the total amount of organic and/or inorganic acids contained in the agent (a) does not exceed a certain value.

In a further suitable embodiment, a process as contemplated herein is exemplified in that the total amount of organic acids from the group including citric acid, tartaric acid, malic acid, and lactic acid contained in the agent (a) is below 1.0% by weight, in an exemplary embodiment below 0.7% by weight, such as below 0.5% by weight, such as below 0.1% by weight and in an exemplary embodiment below 0.01% by weight.

In a further suitable embodiment, a process as contemplated herein is exemplified in that the total amount of inorganic acids from the group including hydrochloric acid, sulfuric acid and phosphoric acid contained in the agent (a) is below 1.0% by weight, in an exemplary embodiment below 0.7% by weight, such as below 0.5% by weight, such as below 0.1% by weight and in an exemplary embodiment below 0.01% by weight.

The maximum total amounts of the acids contained in the agent (a) given above are always based on the total weight of the agent (a).

### Agent (b)

The method of treating keratin material as contemplated herein includes, in addition to the application of agent (a), the application of agent (b). The agent (b) is exemplified in that it includes at least one film-forming polymer (b1).

Polymers are macromolecules with a molecular weight of at least 1000 g/mol, in an exemplary embodiment of at least 2500 g/mol, in an exemplary embodiment of at least 5000 g/mol, which include identical, repeating organic units. The polymers of the present disclosure may be synthetically produced polymers which are manufactured by polymerization of one type of monomer or by polymerization of different types of monomer which are structurally different from each other. If the polymer is produced by polymerizing a type of monomer, it is called a homo-polymer. If structurally different monomer types are used in polymerization, the resulting polymer is called a copolymer.

The maximum molecular weight of the polymer depends on the degree of polymerization (number of polymerized monomers) and the batch size and is determined by the polymerization method. For the purposes of the present invention, it is suitable that the maximum molecular weight of the film-forming hydrophobic polymer (c) is not more than 107 g/mol, in an exemplary embodiment not more than 106 g/mol and in an exemplary embodiment not more than 105 g/mol.

As contemplated herein, a film-forming polymer is a polymer which can form a film on a substrate, for example on a keratinic material or a keratinic fiber. The formation of a film can be demonstrated, for example, by looking at the keratin material treated with the polymer under a microscope.

The film-forming polymers (b1) in the agent (b) can be hydrophilic or hydrophobic.

In a first embodiment, it may be suitable to use at least one hydrophobic film-forming polymer in agent (b).

A hydrophobic polymer is a polymer that has a solubility in water at 25° C. (760 mmHg) of less than 1% by weight.

The water solubility of the film-forming, hydrophobic polymer can be determined in the following way, for example. 1.0 g of the polymer is placed in a beaker. Make up to 100 g with water. A stir-fish is added, and the mixture is heated to 25° C. on a magnetic stirrer while stirring. It is stirred for 60 minutes. The aqueous mixture is then visually assessed. If the polymer-water mixture cannot be assessed visually due to a high turbidity of the mixture, the mixture is filtered. If a proportion of undissolved polymer remains on the filter paper, the solubility of the polymer is less than 1% by weight.

These include acrylic acid-type polymers, polyurethanes, polyesters, polyamides, polyureas, cellulose polymers, nitrocellulose polymers, silicone polymers, acrylamide-type polymers, and polyisoprenes.

Particularly well suited film-forming, hydrophobic polymers are, for example, polymers from the group of copolymers of acrylic acid, copolymers of methacrylic acid, homopolymers or copolymers of acrylic acid esters, homopolymers or copolymers of methacrylic acid esters, homopolymers or copolymers of acrylic acid amides, homopolymers or copolymers of methacrylic acid amides, copolymers of vinylpyrrolidone, copolymers of vinyl alcohol, copolymers of vinyl acetate, homopolymers or copolymers of ethylene, homopolymers or copolymers of propylene, homopolymers or copolymers of styrene, polyurethanes, polyesters and/or polyamides.

In a further suitable embodiment, an agent as contemplated herein is exemplified in that it contains at least one film-forming hydrophobic polymer (c) selected from the group including copolymers of acrylic acid, copolymers of methacrylic acid, homopolymers or copolymers of acrylic acid esters, homopolymers or copolymers of methacrylic acid esters, homopolymers or copolymers of acrylic acid amides, homopolymers or copolymers of methacrylic acid amides, copolymers of vinylpyrrolidone, copolymers of vinyl alcohol, copolymers of vinyl acetate, homopolymers or copolymers of ethylene, homopolymers or copolymers of propylene, homopolymers or copolymers of styrene, polyurethanes, polyesters and/or polyamides.

The film-forming hydrophobic polymers, which are selected from the group of synthetic polymers, polymers obtainable by radical polymerization or natural polymers, have proved to be particularly suitable for solving the problem as contemplated herein.

Other particularly well-suited film-forming hydrophobic polymers can be selected from the homopolymers or copolymers of olefins, such as cycloolefins, butadiene, isoprene or styrene, vinyl ethers, vinyl amides, the esters, or amides of (meth)acrylic acid having at least one C₁-C₂₀ alkyl group, an aryl group or a C₂-C₁₀ hydroxyalkyl group.

Other film-forming hydrophobic polymers may be selected from the homo- or copolymers of isooctyl (meth)acrylate; isonononyl (meth)acrylate; 2-ethylhexyl (meth)acrylate; lauryl (meth)acrylate; isopentyl (meth)acrylate; n-butyl (meth)acrylate); isobutyl (meth)acrylate; ethyl (meth)acrylate; methyl (meth)acrylate; tert-butyl (meth)acrylate; stearyl (meth)acrylate; hydroxyethyl (meth)acrylate; 2-hydroxypropyl (meth)acrylate; 3-hydroxypropyl (meth)acrylate and/or mixtures thereof.

Other film-forming hydrophobic polymers may be selected from the homo- or copolymers of (meth)acrylamide; N-alkyl-(meth)acrylamides, in those with C₂-C₁₈ alkyl groups, such as N-ethyl-acrylamide, N-tert-butyl-acrylamide, le N-octyl-crylamide; N-di(C₁-C₄)alkyl-(meth)acrylamide.

Other suitable anionic copolymers are, for example, copolymers of acrylic acid, methacrylic acid or their C₁-C₆ alkyl esters, as they are marketed under the INCI Declaration Acrylates Copolymers. A suitable commercial product is for example Aculyn® 33 from Rohm & Haas. Copolymers of acrylic acid, methacrylic acid or their C₁-C₆ alkyl esters and the esters of an ethylenically unsaturated acid and an alkoxylated fatty alcohol are also suitable. Suitable ethylenically unsaturated acids are especially acrylic acid, methacrylic acid and itaconic acid; suitable alkoxylated fatty alcohols are especially steareth-20 or ceteth-20.

Very particularly suitable polymers on the market are, for example, Aculyn® 22 (Acrylates/Steareth-20 Methacrylate Copolymer), Aculyn® 28 (Acrylates/Beheneth-25 Methacrylate Copolymer), Structure 20010 (Acrylates/Steareth-20 Itaconate Copolymer), Structure 30010 (Acrylates/Ceteth-20 Itaconate Copolymer), Structure Plus® (Acrylates/Aminoacrylates C10-30 Alkyl PEG-20 Itaconate Copolymer), Carbopol® 1342, 1382, Ultrez 20, Ultrez 21 (Acrylates/C10-30 Alkyl Acrylate Crosspolymer), Synthalen W 2000® (Acrylates/Palmeth-25 Acrylate Copolymer) or the Rohme and Haas distributed Soltex OPT (Acrylates/C12-22 Alkyl methacrylate Copolymer).

The homo- and copolymers of N-vinylpyrrolidone, vinylcaprolactam, vinyl-(C1-C6)alkyl-pyrrole, vinyl-oxazole, vinyl-thiazole, vinylpyrimidine, vinylimidazole can be named as suitable polymers based on vinyl monomers.

Furthermore, the copolymers octylacrylamide/acrylates/butylaminoethyl-methacrylate copolymer, as commercially marketed under the trade names AMPHOMER® or LOVOCRYL® 47 by NATIONAL STARCH, or the copolymers of acrylates/octylacrylamides marketed under the trade names DERMACRYL® LT and DERMACRYL® 79 by NATIONAL STARCH are particularly suitable.

Suitable olefin-based polymers include homopolymers and copolymers of ethylene, propylene, butene, isoprene and butadiene.

In another embodiment, the film-forming hydrophobic polymers may be the block copolymers comprising at least one block of styrene or the derivatives of styrene. These block copolymers can be copolymers that contain one or more other blocks in addition to a styrene block, such as styrene/ethylene, styrene/ethylene/butylene, styrene/butylene, styrene/isoprene, styrene/butadiene. Such polymers are commercially distributed by BASF under the trade name “Luvitol HSB”.

Surprisingly, it was found that particularly intense and washfast colorations could be obtained when agent (b) contained at least one film-forming polymer (b1) selected from the group including acrylic acid homopolymers and copolymers, methacrylic acid homopolymers and copolymers, acrylic acid ester homopolymers and copolymers, methacrylic acid ester homopolymers and copolymers, homopolymers and copolymers of acrylic acid amides, homopolymers and copolymers of methacrylic acid amides, homopolymers and copolymers of vinylpyrrolidone, homopolymers and copolymers of vinyl alcohol, homopolymers and copolymers of vinyl acetate, homopolymers and copolymers of ethylene, homopolymers and copolymers of propylene, homopolymers and copolymers of styrene, polyurethanes, polyesters and polyamides.

In a further suitable embodiment, a process as contemplated herein is exemplified in that the agent (b) includes at least one film-forming polymer (b1) which is selected from the group including the homopolymers and copolymers of acrylic acid, the homopolymers and copolymers of methacrylic acid, the homopolymers and copolymers of acrylic acid esters, the homopolymers and copolymers of methacrylic acid esters homopolymers and copolymers of acrylic acid amides, homopolymers and copolymers of methacrylic acid amides, homopolymers and copolymers of vinylpyrrolidone, homopolymers and copolymers of vinyl alcohol, homopolymers and copolymers of vinyl acetate, homopolymers and copolymers of ethylene, homopolymers and copolymers of propylene, homopolymers and copolymers of styrene, polyurethanes, polyesters and polyamides.

In a further embodiment, it may be suitable to use at least one hydrophilic film-forming polymer (b1) in the agent (b).

A hydrophilic polymer is a polymer that has a solubility in water at 25° C. (760 mmHg) of more than 1% by weight, in an exemplary embodiment more than 2% by weight.

The water solubility of the film-forming, hydrophilic polymer can be determined in the following way, for example. 1.0 g of the polymer is placed in a beaker. Make up to 100 g with water. A stir-fish is added, and the mixture is heated to 25° C. on a magnetic stirrer while stirring. It is stirred for 60 minutes. The aqueous mixture is then visually assessed. A completely dissolved polymer appears macroscopically homogeneous. If the polymer-water mixture cannot be assessed visually due to a high turbidity of the mixture, the mixture is filtered. If no undissolved polymer remains on the filter paper, the solubility of the polymer is more than 1% by weight.

Nonionic, anionic, and cationic polymers can be used as film-forming, hydrophilic polymers.

Suitable film-forming hydrophilic polymers can be selected, for example, from the group of polyvinylpyrrolidone (co)polymers, polyvinyl alcohol (co)polymers, vinyl acetate (co)polymers, carboxyvinyl (co)polymers, acrylic acid (co)polymers, methacrylic acid (co)polymers, natural gums, polysaccharides and/or acrylamide (co)polymers.

Furthermore, it is particularly suitable to use polyvinylpyrrolidone (PVP) and/or a vinylpyrrolidone-containing copolymer as film-forming hydrophilic polymer.

In another particularly suitable embodiment, an agent as contemplated herein is exemplified in that it contains (c) at least one film-forming, hydrophilic polymer selected from the group including polyvinylpyrrolidone (PVP) and the copolymers of polyvinylpyrrolidone.

It is further suitable if the agent as contemplated herein contains polyvinylpyrrolidone (PVP) as the film-forming hydrophilic polymer. Surprisingly, the wash fastness of the dyeing's obtained with agents containing PVP (b9 was also particularly good.

Particularly well-suited polyvinylpyrrolidones are available, for example, under the name Luviskol® K from BASF SE, especially Luviskol® K 90 or Luviskol® K 85 from BASF SE.

The polymer PVP K30, which is marketed by Ashland (ISP, POI Chemical), can also be used as another explicitly very well suited polyvinylpyrrolidone (PVP). PVP K 30 is a polyvinylpyrrolidone which is highly soluble in cold water and has the CAS number 9003-39-8. The molecular weight of PVP K 30 is about 40000 g/mol.

Other particularly suitable polyvinylpyrrolidones are the substances known under the trade names LUVITEC K 17, LUVITEC K 30, LUVITEC K 60, LUVITEC K 80, LUVITEC K 85, LUVITEC K 90 and LUVITEC K 115 and available from BASF.

The use of film-forming hydrophilic polymers (b1) from the group of copolymers of polyvinylpyrrolidone has also led to particularly good and washfast color results.

Vinylpyrrolidone-vinyl ester copolymers, such as those marketed under the trademark Luviskol® (BASF), are particularly suitable film-forming hydrophilic polymers. Luviskol® VA 64 and Luviskol® VA 73, both vinylpyrrolidone/vinyl acetate copolymers, are particularly suitable non-ionic polymers.

Of the vinylpyrrolidone-containing copolymers, a styrene/VP copolymer and/or a vinylpyrrolidone-vinyl acetate copolymer and/or a VP/DMAPA acrylates copolymer and/or a VP/vinyl caprolactam/DMAPA acrylates copolymer are particularly suitable in cosmetic compositions.

Vinylpyrrolidone-vinyl acetate copolymers are marketed under the name Luviskol® VA by BASF SE. For example, a VP/Vinyl Caprolactam/DMAPA Acrylates copolymer is sold under the trade name Aquaflex® SF-40 by Ashland Inc. For example, a VP/DMAPA acrylates copolymer is marketed by Ashland under the name Styleze CC-10 and is a highly suitable vinylpyrrolidone-containing copolymer.

Other suitable copolymers of polyvinylpyrrolidone may also be those obtained by reacting N-vinylpyrrolidone with at least one further monomer from the group including V-vinylformamide, vinyl acetate, ethylene, propylene, acrylamide, vinylcaprolactam, vinylcaprolactone and/or vinyl alcohol.

In another very particularly suitable embodiment, an agent as contemplated herein is exemplified in that it includes at least one film-forming, hydrophilic polymer (b1) selected from the group including polyvinylpyrrolidone (PVP), vinylpyrrolidone/vinyl acetate copolymers, vinylpyrrolidone/styrene copolymers, vinylpyrrolidone/ethylene copoylmeres, vinylpyrrolidone/propylene copolymers, vinylpyrrolidone/vinylcaprolactam copolymers, vinylpyrrolidone/vinylformamide copolymers and/or vinylpyrrolidone/vinyl alcohol copolymers.

Another fussy copolymer of vinylpyrrolidone is the polymer known under the INCI designation maltodextrin/VP copolymer.

Furthermore, intensively dyed keratin material, especially hair, with particularly good wash fastness could be obtained if a non-ionic, film-forming, hydrophilic polymer was used as the film-forming, hydrophilic polymer.

In another embodiment, the agent (b) may contain at least one nonionic, film-forming, hydrophilic polymer (b1).

As contemplated herein, a non-ionic polymer is understood to be a polymer which in a protic solvent—such as water—under standard conditions does not carry structural units with permanent cationic or anionic groups, which must be compensated by counterions while maintaining electron neutrality. Cationic groups include quaternized ammonium groups but not protonated amines Anionic groups include carboxylic and sulphonic acid groups.

Preference is given to products containing, as a non-ionic, film-forming, hydrophilic polymer, at least one polymer selected from the group including
Polyvinylpyrrolidone,
Copolymers of N-vinylpyrrolidone and vinyl esters of carboxylic acids having 2 to 18 carbon atoms, of N-vinylpyrrolidone and vinyl acetate,
Copolymers of N-vinylpyrrolidone and N-vinylimidazole and methacrylamide,
Copolymers of N-vinylpyrrolidone and N-vinylimidazole and acrylamide,
Copolymers of N-vinylpyrrolidone with N,N-di(C1 to C4)-alkylamino-(C2 to C4)-alkylacrylamide,

If copolymers of N-vinylpyrrolidone and vinyl acetate are used, it is again preferable if the molar ratio of the structural units contained in the monomer N-vinylpyrrolidone to the structural units of the polymer contained in the monomer vinyl acetate is in the range from about 20:80 to about 80:20, in particular from about 30:70 to about 60:40. Suitable copolymers of vinyl pyrrolidone and vinyl acetate are available, for example, under the trademarks Luviskol® VA 37, Luviskol® VA 55, Luviskol® VA 64 and Luviskol® VA 73 from BASF SE.

Another particularly suitable polymer is selected from the INCI designation VP/Methacrylamide/Vinyl Imidazole Copolymer, which is available under the trade name Luviset Clear from BASF SE.

Another particularly suitable non-ionic, film-forming, hydrophilic polymer is a copolymer of N-vinylpyrrolidone and N,N-dimethylaminiopropylmethacrylamide, which is sold under the INCI designation VP/DMAPA Acrylates Copolymer e.g., under the trade name Styleze® CC 10 by ISP.

A cationic polymer of interest is the copolymer of N-vinylpyrrolidone, N-vinylcaprolactam, N-(3-dimethylaminopropyl)methacrylamide and 3-(methacryloylamino)propyl-lauryl-dimethylammonium chloride (INCI designation): Polyquaternium-69), which is marketed, for example, under the trade name AquaStyle® 300 (28-32 wt. % active substance in ethanol-water mixture, molecular weight 350000) by ISP.

Other suitable film-forming, hydrophilic polymers include
Vinylpyrrolidone-vinylimidazolium methochloride copolymers, as offered under the designations Luviquat® FC 370, FC 550 and the INCI designation Polyquaternium-16 as well as FC 905 and HM 552,
Vinylpyrrolidone-vinylcaprolactam-acrylate terpolymers, as they are commercially available with acrylic acid esters and acrylic acid amides as a third monomer component, for example under the name Aquaflex® SF 40.

Polyquaternium-11 is the reaction product of diethyl sulphate with a copolymer of vinyl pyrrolidone and dimethylaminoethyl methacrylate. Suitable commercial products are available under the names Dehyquart® CC 11 and Luviquat® PQ 11 PN from BASF SE or Gafquat 440, Gafquat 734, Gafquat 755 or Gafquat 755N from Ashland Inc.

Polyquaternium-46 is the reaction product of vinylcaprolactam and vinylpyrrolidone with methylvinylimidazolium methosulfate and is available for example under the name Luviquat® Hold from BASF SE. Polyquaternium-46 is in an exemplary embodiment used in an amount of 1 to 5% by weight—based on the total weight of the cosmetic composition. It particularly prefers to use polyquaternium-46 in combination with a cationic guar compound. It is even highly suitable that polyquaternium-46 is used in combination with a cationic guar compound and polyquaternium-11.

Suitable anionic film-forming, hydrophilic polymers can be, for example, acrylic acid polymers, which can be in non-crosslinked or crosslinked form. Such products are sold commercially under the trade names Carbopol 980, 981, 954, 2984 and 5984 by Lubrizol or under the names Synthalen M and Synthalen K by 3V Sigma (The Sun Chemicals, Inter Harz).

Examples of suitable film-forming, hydrophilic polymers from the group of natural gums are xanthan gum, gellan gum, carob gum.

Examples of suitable film-forming hydrophilic polymers from the group of polysaccharides are hydroxyethyl cellulose, hydroxypropyl cellulose, ethyl cellulose and carboxymethyl cellulose.

Suitable film-forming, hydrophilic polymers from the group of acrylamdes are, for example, polymers which are produced from monomers of (methy)acrylamido-C1-C4-alkyl sulphonic acid or the salts thereof. Corresponding polymers may be selected from the polymers of polyacrylamidomethanesulfonic acid, polyacrylamidoethanesulfonic acid, polyacrylamidopropanesulfonic acid, poly2-acrylamido-2-methylpropanesulfonic acid, poly-2-methylacrylamido-2-methylpropanesulfonic acid and/or poly-2-methylacrylamido-n-butanesulfonic acid.

Suitable polymers of the poly(meth)arylamido-C1-C4-alkyl sulphonic acids are cross-linked and at least 90% neutralized. These polymers can or cannot be cross-linked.

Cross-linked and fully or partially neutralized polymers of the poly-2-acrylamido-2-methylpropane sulfonic acid type are available under the INCI designation “Ammonium Polyacrylamido-2-methyl-propanesulphonates” or “Ammonium Polyacryldimethyltauramides”.

Another suitable polymer of this type is the cross-linked poly-2-acrylamido-2-methyl-propanesulphonic acid polymer marketed by Clamant under the trade name Hostacerin AMPS, which is partially neutralized with ammonia.

In a further explicitly quite particularly suitable embodiment, a process as contemplated herein is exemplified in that the agent (b) includes at least one anionic, film-forming, polymer (b1).

In this context, the best results were obtained when the agent (b) contains at least one film-forming polymer (b1) comprising at least one structural unit of formula (P-I) and at least one structural unit of formula (P-II)

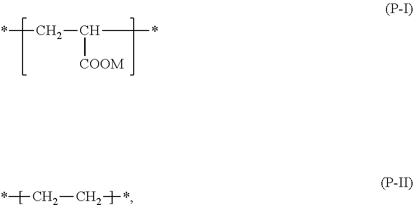

where
M is a hydrogen atom or ammonium (NH₄), sodium, potassium, ½ magnesium or ½ calcium.

In a further suitable embodiment, a process as contemplated herein is exemplified in that the agent (b) includes at least one film-forming polymer (b1) which includes at least one structural unit of the formula (P-I) and at least one structural unit of the formula (P-II)

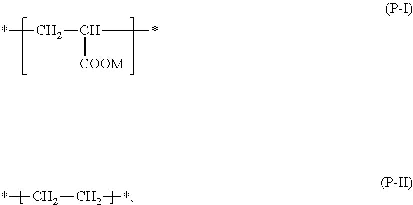

where
M is a hydrogen atom or ammonium (NH₄), sodium, potassium, ½ magnesium or ½ calcium.

The film-forming polymer or polymers (b1) as contemplated herein are in an exemplary embodiment used in certain ranges of amounts in the agent (b) as contemplated herein. In this context, it has proved particularly preferable for solving the problem as contemplated herein if the agent (b) contains—based on the total weight of the agent (b)—one or more film-forming polymers (b1) in a total amount of from about 0.1 to about 18.0% by weight, in an exemplary embodiment from about 1.0 to about 16.0% by weight, such as from about 5.0 to about 14.5% by weight and in an exemplary embodiment from about 8.0 to about 12.0% by weight.

In a further suitable embodiment, a process as contemplated herein is exemplified in that the agent (b) contains—based on the total weight of the agent (b)—one or more film-forming polymers (b1) in a total amount of from about 0.1 to about 18.0% by weight, in an exemplary embodiment from about 1.0 to about 16.0% by weight, such as from about 5.0 to about 14.5% by weight and in an exemplary embodiment from about 8.0 to about 12.0% by weight.

### Agent for Dyeing Keratin Material

By using the agent (a) as described above, the organic silicon compound (a1) comprising one or more hydroxyl groups or hydrolyzable groups per molecule are first hydrolyzed and/or oligomerized in the presence of the water. The resulting hydrolysis products or oligomers have a particularly high affinity to the surface of the Keratin material. The presence of the alkalizing agents (a2) in the agent (a) accelerates or optimizes this film formation. Following the application of agent (a), agent (b) is now applied, whereby the film-forming polymers (b1) contained in this agent (b) are deposited on the keratinous material in the form of a second film. The successive application of agents (a) and (b) thus creates a layering of several films that is particularly resistant to external influences.

This formation of the multilayer film system is of advantage when coloring the keratinous material. If the agent (a) and/or (b) additionally contains a colorant compound from the group of pigments or direct dyes, colored films are produced in this way. The colorant compounds trapped in these resistant films exhibit extremely good wash fastness.

In an explicitly quite particularly suitable embodiment, a process as contemplated herein is exemplified in that the agent (a) and/or the agent (b) contains at least one colorant compound from the group including pigments and/or direct dyes.

In other words, explicitly quite particularly suitable is a method for coloring keratinous material, in particular human hair, comprising the following steps:
Application of a water-containing agent (a) to the keratinous material, wherein the agent (a) has and contains a pH of at least 9.6:
(a1) at least one organic silicon compound selected from the group including silanes having one, two or three silicon atoms, and
(a2) at least one alkalizing agent selected from the group including ammonia, alkanolamines and basic amino acids, and
Application of an agent (b) to the keratinous material, wherein the agent (b) includes:
(b1) at least one film-forming polymer,
   exemplified in that the agent (a) and/or the agent (b) contains at least one colorant compound selected from the group including pigments and/or direct dyes.

The term “coloring agent” is used in the context of the present disclosure for a coloring of the keratin material, of the hair, caused using pigments and/or direct dyes. In this coloring process, the coloring compounds are deposited on the surface of the keratin material in a particularly homogeneous, uniform, and smooth film. The film is formed in situ by oligomerization or polymerization of the organic silicon compound(s) in an alkaline environment, and by the interaction of organic silicone compound with the colorant compound(s) and the film-forming polymer.

It has been found to be most preferable when the coloring compound is contained in the agent (a).

In other words, explicitly quite particularly suitable is a method for coloring keratinous material, in particular human hair, comprising the following steps:
Application of a water-containing agent (a) to the keratinous material, wherein the agent (a) has and contains a pH of at least 9.6:
(a1) at least one organic silicon compound selected from the group including silanes having one, two or three silicon atoms, and
(a2) at least one alkalizing agent selected from the group including ammonia, alkanolamines and basic amino acids, and
(a3) at least one colorant compound selected from the group including pigments and/or direct dyes, and
Application of an agent (b) to the keratinous material, wherein the agent (b) includes:
(b1) at least one film-forming polymer.

In the context of a further embodiment as contemplated herein is also a method for coloring keratinous material, in particular human hair, comprising the following steps:
Application of a water-containing agent (a) to the keratinous material, wherein the agent (a) has and contains a pH of at least 9.6:
(a1) at least one organic silicon compound selected from the group including silanes having one, two or three silicon atoms, and
(a2) at least one alkalizing agent selected from the group including ammonia, alkanolamines and basic amino acids, and
Application of an agent (b) to the keratinous material, wherein the agent (b) includes:
(b1) at least one film-forming polymer, and
(b2) at least one colorant compound selected from the group including pigments and/or direct dyes.

In a further embodiment, however, the colorant compound can also be applied to the keratin material in the form of a third agent (c).

In the context of this further embodiment, as contemplated herein is also a method for coloring keratinous material, in particular human hair, comprising the following steps:
Application of a water-containing agent (a) to the keratinous material, wherein the agent (a) has and contains a pH of at least 9.6:
(a1) at least one organic silicon compound selected from the group including silanes having one, two or three silicon atoms, and
(a2) at least one alkalizing agent selected from the group including ammonia, alkanolamines and basic amino acids, and
Application of an agent (b) to the keratinous material, wherein the agent (b) includes:
(b1) at least one film-forming polymer, and
Application of an agent (c) to the keratinous material, wherein the agent (c) includes:
(c1) at least one colorant compound selected from the group including pigments and/or direct dyes.
   Coloring Compounds from the Group of Pigments and/or Direct Dyes

In an exemplary embodiment, the process as contemplated herein is used for dyeing keratin material. In this case, the agent (a) and/or the agent (b)—or also a separately prepared third agent (c)—contains at least one colorant compound from the group including pigments and/or direct dyes.

Pigments within the meaning of the present disclosure are coloring compounds which have a solubility in water at 25° C. of less than 0.5 g/L, in an exemplary embodiment less than 0.1 g/L, such as less than 0.05 g/L. Water solubility can be determined, for example, by the method described below: 0.5 g of the pigment are weighed in a beaker. A stir-fish is added. Then one liter of distilled water is added. This mixture is heated to 25° C. for one hour while stirring on a magnetic stirrer. If undissolved components of the pigment are still visible in the mixture after this period, the solubility of the pigment is below 0.5 g/L. If the pigment-water mixture cannot be assessed visually due to the high intensity of the possibly finely dispersed pigment, the mixture is filtered. If a proportion of undissolved pigments remains on the filter paper, the solubility of the pigment is below 0.5 g/L.

Suitable color pigments can be of inorganic and/or organic origin.

In a suitable embodiment, an agent as contemplated herein is exemplified in that the agent (a) and/or (b) (or else a third agent (c)) contains at least one color-imparting compound from the group of inorganic and/or organic pigments.

Suitable color pigments are selected from synthetic or natural inorganic pigments. Inorganic color pigments of natural origin can be produced, for example, from chalk, ochre, umber, green earth, burnt Terra di Siena or graphite. Furthermore, black pigments such as iron oxide black, colored pigments such as ultramarine or iron oxide red as well as fluorescent or phosphorescent pigments can be used as inorganic color pigments.

Particularly suitable are colored metal oxides, hydroxides and oxide hydrates, mixed-phase pigments, sulfur-containing silicates, silicates, metal sulfides, complex metal cyanides, metal sulphates, chromates and/or molybdates. Suitable color pigments are black iron oxide (CI 77499), yellow iron oxide (CI 77492), red and brown iron oxide (CI 77491), manganese violet (CI 77742), ultramarine (sodium aluminum sulfo silicates, CI 77007, pigment blue 29), chromium oxide hydrate (CI77289), iron blue (ferric ferrocyanides, CI77510) and/or carmine (cochineal).

As contemplated herein, colored pearlescent pigments are also particularly suitable color pigments. These are usually mica- and/or mica-based and can be coated with one or more metal oxides. Mica belongs to the layer silicates. The most important representatives of these silicates are muscovite, phlogopite, paragonite, biotite, lepidolite and margarite. To produce the pearlescent pigments in combination with metal oxides, the mica, mainly muscovite or phlogopite, is coated with a metal oxide.

As an alternative to natural mica, synthetic mica coated with one or more metal oxides can also be used as pearlescent pigment. Especially suitable pearlescent pigments are based on natural or synthetic mica (mica) and are coated with one or more of the metal oxides mentioned above. The color of the respective pigments can be varied by varying the layer thickness of the metal oxide(s).

In a further suitable embodiment, a process as contemplated herein is exemplified in that the agent (a) and/or (b) includes at least one colorant compound from the group of pigments selected from the group of colored metal oxides, metal hydroxides, metal oxide hydrates, silicates, metal sulfides, complex metal cyanides, metal sulfates, bronze pigments and/or from colored mica- or mica-based pigments coated with at least one metal oxide and/or a metal oxychloride.

In a further suitable embodiment, an agent as contemplated herein is exemplified in that it contains (b) at least one colorant compound from the group of pigments selected from pigments based on mica or micaceous iron oxide, which is combined with one or more metal oxides from the group of titanium dioxide (CI 77891), are coated with black iron oxide (CI 77499), yellow iron oxide (CI 77492), red and/or brown iron oxide (CI 77491, CI 77499), manganese violet (CI 77742), ultramarine (sodium aluminum sulfo silicates, CI 77007, pigment blue 29), chromium oxide hydrate (CI 77289), chromium oxide (CI 77288) and/or iron blue (ferric ferrocyanides, CI 77510).

Examples of particularly suitable color pigments are commercially available under the trade names Rona®, Colorona®, Xirona®, Dichrona® and Timiron® from Merck, Ariabel® and Unipure® from Sensient, Prestige® from Eckart Cosmetic Colors and Sunshine® from Sunstar.

Particularly suitable color pigments with the trade name Colorona® are, for example:

### Colorona Copper, Merck, MICA, CI 77491 (IRON OXIDES)

### Colorona Passion Orange, Merck, Mica, CI 77491 (Iron Oxides), Alumina

### Colorona Patina Silver, Merck, MICA, CI 77499 (IRON OXIDES), CI 77891 (TITANIUM DIOXIDE)

### Colorona RY, Merck, CI 77891 (TITANIUM DIOXIDE), MICA, CI 75470 (CARMINE)

### Colorona Oriental Beige, Merck, MICA, CI 77891 (TITANIUM DIOXIDE), CI 77491 (IRON OXIDES)

### Colorona Dark Blue, Merck, MICA, TITANIUM DIOXIDE, FERRIC FERROCYANIDE

### Colorona Chameleon, Merck, CI 77491 (IRON OXIDES), MICA

### Colorona Aborigine Amber, Merck, MICA, CI 77499 (IRON OXIDES), CI 77891 (TITANIUM DIOXIDE)

### Colorona Blackstar Blue, Merck, CI 77499 (IRON OXIDES), MICA

### Colorona Patagonian Purple, Merck, MICA, CI 77491 (IRON OXIDES), CI 77891 (TITANIUM DIOXIDE), CI 77510 (FERRIC FERROCYANIDE)

### Colorona Red Brown, Merck, MICA, CI 77491 (IRON OXIDES), CI 77891 (TITANIUM DIOXIDE)

### Colorona Russet, Merck, CI 77491 (TITANIUM DIOXIDE), MICA, CI 77891 (IRON OXIDES)

### Colorona Imperial Red, Merck, MICA, TITANIUM DIOXIDE (CI 77891), D&C RED NO. 30 (CI 73360)

### Colorona Majestic Green, Merck, CI 77891 (TITANIUM DIOXIDE), MICA, CI 77288 (CHROMIUM OXIDE GREENS)

### Colorona Light Blue, Merck, MICA, TITANIUM DIOXIDE (CI 77891), FERRIC FERROCYANIDE (CI 77510)

### Colorona Red Gold, Merck, MICA, CI 77891 (TITANIUM DIOXIDE), CI 77491 (IRON OXIDES)

### Colorona Gold Plus MP 25, Merck, MICA, TITANIUM DIOXIDE (CI 77891), IRON OXIDES (CI 77491)

### Colorona Carmine Red, Merck, MICA, TITANIUM DIOXIDE, CARMINE

### Colorona Blackstar Green, Merck, MICA, CI 77499 (IRON OXIDES)

### Colorona Bordeaux, Merck, MICA, CI 77491 (IRON OXIDES)

### Colorona Bronze, Merck, MICA, CI 77491 (IRON OXIDES)

### Colorona Bronze Fine, Merck, MICA, CI 77491 (IRON OXIDES)

### Colorona Fine Gold MP 20, Merck, MICA, CI 77891 (TITANIUM DIOXIDE), CI 77491 (IRON OXIDES)

### Colorona Sienna Fine, Merck, CI 77491 (IRON OXIDES), MICA

### Colorona Sienna, Merck, MICA, CI 77491 (IRON OXIDES)

Colorona Precious Gold, Merck, Mica, CI 77891 (Titanium dioxide), Silica, CI 77491 (Iron oxides), Tin oxide

### Colorona Sun Gold Sparkle MP 29, Merck, MICA, TITANIUM DIOXIDE, IRON OXIDES, MICA, CI 77891, CI 77491 (EU)

Colorona Mica Black, Merck, CI 77499 (Iron oxides), Mica, CI 77891 (Titanium dioxide)
Colorona Bright Gold, Merck, Mica, CI 77891 (Titanium dioxide), CI 77491 (Iron oxides)

### Colorona Blackstar Gold, Merck, MICA, CI 77499 (IRON OXIDES)

Other particularly suitable color pigments with the trade name Xirona® are for example:

### Xirona Golden Sky, Merck, Silica, CI 77891 (Titanium Dioxide), Tin Oxide

### Xirona Caribbean Blue, Merck, Mica, CI 77891 (Titanium Dioxide), Silica, Tin Oxide

### Xirona Kiwi Rose, Merck, Silica, CI 77891 (Titanium Dioxide), Tin Oxide

### Xirona Magic Mauve, Merck, Silica, CI 77891 (Titanium Dioxide), Tin Oxide.

In addition, particularly suitable color pigments with the trade name Unipure® are for example:

### Unipure Red LC 381 EM, Sensient CI 77491 (Iron Oxides), Silica

### Unipure Black LC 989 EM, Sensient, CI 77499 (Iron Oxides), Silica

### Unipure Yellow LC 182 EM, Sensient, CI 77492 (Iron Oxides), Silica

In a further embodiment, the agent (a) and/or the agent (b) may include one or more colorant compounds selected from the group including organic pigments

The organic pigments as contemplated herein are correspondingly insoluble, organic dyes or color lacquers, which may be selected, for example, from the group of nitroso, nitro-azo, xanthene, anthraquinone, isoindolinone, isoindolinone, quinacridone, perinone, perylene, diketo-pyrrolopyorrole, indigo, thioindido, dioxazine and/or triarylmethane compounds.

Examples of particularly suitable organic pigments are carmine, quinacridone, phthalocyanine, sorghum, blue pigments with the Color Index numbers CI 42090, CI 69800, CI 69825, CI 73000, CI 74100, CI 74160, yellow pigments with the Color Index numbers CI 11680, CI 11710, CI 15985, CI 19140, CI 20040, CI 21100, CI 21108, CI 47000, CI 47005, green pigments with the Color Index numbers CI 61565, CI 61570, CI 74260, orange pigments with the Color Index numbers CI 11725, CI 15510, CI 45370, CI 71105, red pigments with the Color Index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915 and/or CI 75470.

In a further particularly suitable embodiment, a process as contemplated herein is exemplified in that the agent (a) and/or the agent (b) contains at least one colorant compound from the group of organic pigments selected from the group of carmine, quinacridone, phthalocyanine, sorghum, blue pigments with the Color Index numbers CI 42090, CI 69800, CI 69825, CI 73000, CI 74100, CI 74160, yellow pigments with the Color Index numbers CI 11680, CI 11710, CI 15985, CI 19140, CI 20040, CI 21100, CI 21108, CI 47000, CI 47005, green pigments with Color Index numbers CI 61565, CI 61570, CI 74260, orange pigments with Color Index numbers CI 11725, CI 15510, CI 45370, CI 71105, red pigments with the Color Index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915 and/or CI 75470.

The organic pigment can also be a color paint. As contemplated herein, the term color lacquer means particles comprising a layer of absorbed dyes, the unit of particle and dye being insoluble under the above-mentioned conditions. The particles can, for example, be inorganic substrates, which can be aluminum, silica, calcium borosilate, calcium aluminum borosilicate or even aluminum.

For example, alizarin color varnish can be used.

Due to their excellent resistance to light and temperature, the use of the pigments in the means as contemplated herein is particularly suitable. It is also suitable if the pigments used have a certain particle size. This particle size leads on the one hand to an even distribution of the pigments in the formed polymer film and on the other hand avoids a rough hair or skin feeling after application of the cosmetic product. As contemplated herein, it is therefore advantageous if the at least one pigment has an average particle size D50 of about 1.0 to about 50 μm, in an exemplary embodiment about 5.0 to about 45 μm, such as about 10 to about 40 μm, about 14 to about 30 μm. The mean particle size D50D₅₀, for example, can be determined using dynamic light scattering (DLS).

In a further suitable embodiment, a process as contemplated herein is exemplified in that the agent (a) contains—based on the total weight of the agent (a)—one or more pigments in a total amount of from about 0.01 to about 10.0% by weight, in an exemplary embodiment from about 0.1 to about 8.0% by weight, such as from about 0.2 to about 6.0% by weight and in an embodiment from about 0.5 to about 4.5% by weight.

In a further suitable embodiment, a process as contemplated herein is exemplified in that the agent (b) contains—based on the total weight of the agent (b)—one or more pigments in a total amount of from 0.01 to 10.0% by weight, in an exemplary embodiment from about 0.1 to about 8.0% by weight, such as from about 0.2 to about 6.0% by weight and in an embodiment from about 0.5 to about 4.5% by weight.

As colorant compounds, the agents (a) and/or (b) used in the process as contemplated herein (or also in a separately prepared third agent (c)) may also contain one or more direct dyes. Direct-acting dyes are dyes that draw directly onto the hair and do not require an oxidative process to form the color. Direct dyes are usually nitrophenylene diamines, nitroaminophenols, azo dyes, anthraquinones, triarylmethane dyes or indophenols.

The direct dyes within the meaning of the present disclosure have a solubility in water (760 mmHg) at 25° C. of more than 0.5 g/L and are therefore not to be regarded as pigments.

In an exemplary embodiment, the direct dyes within the meaning of the present disclosure have a solubility in water (760 mmHg) at 25° C. of more than 1.0 g/L.

Direct dyes can be divided into anionic, cationic, and non-ionic direct dyes.

In a further suitable embodiment, a process as contemplated herein is exemplified in that the agent (a) and/or the agent (b) contains at least one anionic, cationic and/or nonionic direct dye as the coloring compound (b).

In a further suitable embodiment, a process as contemplated herein is exemplified in that the agent (a) and/or the agent (b) includes at least one anionic, cationic and/or nonionic direct dye.

Suitable cationic direct dyes include Basic Blue 7, Basic Blue 26, Basic Violet 2, and Basic Violet 14, Basic Yellow 57, Basic Red 76, Basic Blue 16, Basic Blue 347 (Cationic Blue 347/Dystar), HC Blue No. 16, Basic Blue 99, Basic Brown 16, Basic Brown 17, Basic Yellow 57, Basic Yellow 87, Basic Orange 31, Basic Red 51 Basic Red 76.

As non-ionic direct dyes, non-ionic nitro and quinone dyes and neutral azo dyes can be used. Suitable non-ionic direct dyes are those listed under the international designations or Trade names HC Yellow 2, HC Yellow 4, HC Yellow 5, HC Yellow 6, HC Yellow 12, HC Orange 1, Disperse Orange 3, HC Red 1, HC Red 3, HC Red 10, HC Red 11, HC Red 13, HC Red BN, HC Blue 2, HC Blue 11, HC Blue 12, Disperse Blue 3, HC Violet 1, Disperse Violet 1, Disperse Violet 4, Disperse Black 9 known compounds, as well as 1,4-diamino-2-nitrobenzene, 2-amino-4-nitrophenol, 1,4-bis-(2-hydroxyethyl)-amino-2-nitrobenzene, 3-nitro-4-(2-hydroxyethyl)-aminophenol 2-(2-hydroxyethyl)amino-4,6-dinitrophenol, 4-[(2-hydroxyethyl)amino]-3-nitro-1-methylbenzene, 1-amino-4-(2-hydroxyethyl)-amino-5-chloro-2-nitrobenzene, 4-amino-3-nitrophenol, 1-(2′-ureidoethyl)amino-4-nitrobenzene, 2-[(4-amino-2-nitrophenyl)amino]benzoic acid, 6-nitro-1,2,3,4-tetrahydroquinoxaline, 2-hydroxy-1,4-naphthoquinone, picramic acid and its salts, 2-amino-6-chloro-4-nitrophenol, 4-ethylamino-3-nitrobenzoic acid and 2-chloro-6-ethylamino-4-nitrophenol.

In the course of the work leading to the present invention, it has been found that dyeing's of particularly high color intensity can be produced with agents (a) and/or (b) containing at least one anionic direct dye.

In an explicitly quite particularly suitable embodiment, a process as contemplated herein is therefore exemplified in that the agent (a) and/or the agent (b) includes at least one anionic direct dye.

Anionic direct dyes are also called acid dyes. Acid dyes are direct dyes that have at least one carboxylic acid group (—COOH) and/or one sulphonic acid group (—SO₃H). Depending on the pH value, the protonated forms (—COOH, —SO₃H) of the carboxylic acid or sulphonic acid groups are in equilibrium with their deprotonated forms (—OO⁻, —SO₃— present). The proportion of protonated forms increases with decreasing pH. If direct dyes are used in the form of their salts, the carboxylic acid groups or sulphonic acid groups are present in deprotonated form and are neutralized with corresponding stoichiometric equivalents of cations to maintain electro neutrality. Inventive acid dyes can also be used in the form of their sodium salts and/or their potassium salts.

The acid dyes within the meaning of the present disclosure have a solubility in water (760 mmHg) at 25° C. of more than 0.5 g/L and are therefore not to be regarded as pigments. In an exemplary embodiment the acid dyes within the meaning of the present disclosure have a solubility in water (760 mmHg) at 25° C. of more than 1.0 g/L.

The alkaline earth salts (such as calcium salts and magnesium salts) or aluminum salts of acid dyes often have a lower solubility than the corresponding alkali salts. If the solubility of these salts is below 0.5 g/L (25° C., 760 mmHg), they do not fall under the definition of a direct dye.

An essential characteristic of acid dyes is their ability to form anionic charges, whereby the carboxylic acid or sulphonic acid groups responsible for this are usually linked to different chromophoric systems. Suitable chromophoric systems can be found, for example, in the structures of nitrophenylenediamines, nitroaminophenols, azo dyes, anthraquinone dyes, triarylmethane dyes, xanthene dyes, rhodamine dyes, oxazine dyes and/or indophenol dyes.

In one embodiment, a process for dyeing keratinous material is thus suitable, which is exemplified in that the agent (a) and/or the agent (b) includes at least one anionic direct dye selected from the group including nitrophenylenediamines, nitroaminophenols, azo dyes, anthraquinone dyes, triarylmethane dyes, xanthene dyes, rhodamine dyes, oxazine dyes and/or indophenol dyes, the xanthene dyes, the rhodamine dyes, the oxazine dyes and/or the indophenol dyes, the dyes from the abovementioned group each having at least one carboxylic acid group (—COOH), a sodium carboxylate group (—COONa), a potassium carboxylate group (—COOK), a sulfonic acid group (—SO₃H), a sodium sulfonate group (—SO₃Na) and/or a potassium sulfonate group (—SO₃K).

For example, one or more compounds from the following group can be selected as particularly well suited acid dyes: Acid Yellow 1 (D&C Yellow 7, Citronin A, Ext. D&C Yellow No. 7, Japan Yellow 403, CI 10316, COLIPA no B001), Acid Yellow 3 (COLIPA no: C 54, D&C Yellow No 10, Quinoline Yellow, E104, Food Yellow 13), Acid Yellow 9 (CI 13015), Acid Yellow 17 (CI 18965), Acid Yellow 23 (COLIPA no C. 29, Covacap Jaune W 1100 (LCW), Sicovit Tartrazine 85 E 102 (BASF), Tartrazine, Food Yellow 4, Japan Yellow 4, FD&C Yellow No. 5), Acid Yellow 36 (CI 13065), Acid Yellow 121 (CI 18690), Acid Orange 6 (CI 14270), Acid Orange 7 (2-Naphthol orange, Orange II, CI 15510, D&C Orange 4, COLIPA no C015), Acid Orange 10 (C.I. 16230; Orange G sodium salt), Acid Orange 11 (CI 45370), Acid Orange 15 (CI 50120), Acid Orange 20 (CI 14600), Acid Orange 24 (BROWN 1; CI 20170; KATSU201; nosodiumsalt; Brown No. 201; RESORCIN BROWN; ACID ORANGE 24; Japan Brown 201; D & C Brown No. 1), Acid Red 14 (C.I. 14720), Acid Red 18 (E124, Red 18; CI 16255), Acid Red 27 (E 123, CI 16185, C-Rot 46, Echtrot D, FD&C Red Nr. 2, Food Red 9, Naphtholrot S), Acid Red 33 (Red 33, Fuchsia Red, D&C Red 33, CI 17200), Acid Red 35 (CI C.I. 18065), Acid Red 51 (CI 45430, Pyrosin B, Tetraiodfluorescein, Eosin J, Iodeosin), Acid Red 52 (CI 45100, Food Red 106, Solar Rhodamine B, Acid Rhodamine B, Red n° 106 Pontacyl Brilliant Pink), Acid Red 73 (CI 27290), Acid Red 87 (Eosin, CI 45380), Acid Red 92 (COLIPA no C53, CI 45410), Acid Red 95 (CI 45425, Erythtosine, Simacid Erythrosine Y), Acid Red 184 (CI 15685), Acid Red 195, Acid Violet 43 (Jarocol Violet 43, Ext. D&C Violet no 2, C.I. 60730, COLIPA no C063), Acid Violet 49 (CI 42640), Acid Violet 50 (CI 50325), Acid Blue 1 (Patent Blue, CI 42045), Acid Blue 3 (Patent Blue V, CI 42051), Acid Blue 7 (CI 42080), Acid Blue 104 (CI 42735), Acid Blue 9 (E 133, Patent blue AE, Amidoblau AE, Erioglaucin A, CI 42090, C.I. Food Blue 2), Acid Blue 62 (CI 62045), Acid Blue 74 (E 132, CI 73015), Acid Blue 80 (CI 61585), Acid Green 3 (CI 42085, Foodgreen1), Acid Green 5 (CI 42095), Acid Green 9 (C.I. 42100), Acid Green 22 (C.I. 42170), Acid Green 25 (CI 61570, Japan Green 201, D&C Green No. 5), Acid Green 50 (Brilliant Acid Green BS, C.I. 44090, Acid Brilliant Green BS, E 142), Acid Black 1 (Black n° 401, Naphthalene Black 10B, Amido Black 10B, CI 20 470, COLIPA no B15), Acid Black 52 (CI 15711), Food Yellow 8 (CI 14270), Food Blue 5, D&C Yellow 8, D&C Green 5, D&C Orange 10, D&C Orange 11, D&C Red 21, D&C Red 27, D&C Red 33, D&C Violet 2 and/or D&C Brown 1.

For example, the water solubility of anionic direct dyes can be determined in the following way. 0.1 g of the anionic direct dye is placed in a beaker. A stir-fish is added. Then add 100 ml of water. This mixture is heated to 25° C. on a magnetic stirrer while stirring. It is stirred for 60 minutes. The aqueous mixture is then visually assessed. If there are still undissolved residues, the amount of water is increased—for example in steps of 10 ml. Water is added until the amount of dye used is completely dissolved. If the dye-water mixture cannot be assessed visually due to the high intensity of the dye, the mixture is filtered. If a proportion of undissolved dyes remains on the filter paper, the solubility test is repeated with a higher quantity of water. If 0.1 g of the anionic direct dye dissolves in 100 ml water at 25° C., the solubility of the dye is 1.0 g/L.

Acid Yellow 1 is called 8-hydroxy-5,7-dinitro-2-naphthalenesulfonic acid disodium salt and has a solubility in water of at least 40 g/L (25° C.).

Acid Yellow 3 is a mixture of the sodium salts of mono- and sisulfonic acids of 2-(2-quinolyl)-1H-indene-1,3(2H)-dione and has a water solubility of 20 g/L (25° C.).
Acid Yellow 9 is the disodium salt of 8-hydroxy-5,7-dinitro-2-naphthalenesulfonic acid, its solubility in water is above 40 g/L (25° C.).
Acid Yellow 23 is the trisodium salt of 4,5-dihydro-5-oxo-1-(4-sulfophenyl)-4-((4-sulfophenyl)azo)-1H-pyrazole-3-carboxylic acid and is highly soluble in water at 25° C.
Acid Orange 7 is the sodium salt of 4-[(2-hydroxy-1-naphthyl)azo]benzene sulphonate. Its water solubility is more than 7 g/L (25° C.).
Acid Red 18 is the trinatirum salt of 7-hydroxy-8-[(E)-(4-sulfonato-1-naphthyl)-diazenyl)]-1,3-naphthalene disulfonate and has a very high-water solubility of more than 20% by weight.
Acid Red 33 is the diantrium salt of 5-amino-4-hydroxy-3-(phenylazo)-naphthalene-2,7-disulphonate, its solubility in water is 2.5 g/L (25° C.).
Acid Red 92 is the disodium salt of 3,4,5,6-tetrachloro-2-(1,4,5,8-tetrabromo-6-hydroxy-3-oxoxanthen-9-yl)benzoic acid, whose solubility in water is indicated as greater than 10 g/L (25° C.).
Acid Blue 9 is the disodium salt of 2-({4-[N-ethyl(3-sulfonatobenzyl]amino]phenyl}{4-[(N-ethyl(3-sulfonatobenzyl)imino]-2,5-cyclohexadien-1-ylidene}methyl)-benzenesulfonate and has a solubility in water of more than 20% by weight (25° C.).

A very particularly suitable process as contemplated herein is therefore exemplified in that the agent (a) and/or the agent (b) includes at least one direct dye selected from the group including Acid Yellow 1, Acid Yellow 3, Acid Yellow 9, Acid Yellow 17, Acid Yellow 23, Acid Yellow 36, Acid Yellow 121, Acid Orange 6, Acid Orange 7, Acid Orange 10, Acid Orange 11, Acid Orange 15, Acid Orange 20, Acid Orange 24, Acid Red 14, Acid Red, Acid Red 27, Acid Red 33, Acid Red 35, Acid Red 51, Acid Red 52, Acid Red 73, Acid Red 87, Acid Red 92, Acid Red 95, Acid Red 184, Acid Red 195, Acid Violet 43, Acid Violet 49, Acid Violet 50, Acid Blue 1, Acid Blue 3, Acid Blue 7, Acid Blue 104, Acid Blue 9, Acid Blue 62, Acid Blue 74, Acid Blue 80, Acid Green 3, Acid Green 5, Acid Green 9, Acid Green 22, Acid Green 25, Acid Green 50, Acid Black 1, Acid Black 52, Food Yellow 8, Food Blue 5, D&C Yellow 8, D&C Green 5, D&C Orange 10, D&C Orange 11, D&C Red 21, D&C Red 27, D&C Red 33, D&C Violet 2 and/or D&C Brown 1.

The direct dye(s), in the anionic direct dyes, can be used in different amounts in the respective (a) and/or (b) agent depending on the desired color intensity. Particularly good results could be obtained if the respective agent—based on its total weight—contains one or more direct dyes (b) in a total amount of about 0.01 to about 10.0% by weight, in an exemplary embodiment from about 0.1 to about 8.0% by weight, such as from about 0.2 to about 6.0% by weight and for example from about 0.5 to about 4.5% by weight.

In a further suitable embodiment, a process as contemplated herein is exemplified in that the agent (a) contains—based on the total weight of the agent (a)—one or more direct dyes in a total amount of from about 0.01 to about 10.0% by weight, in an exemplary embodiment from about 0.1 to about 8.0% by weight, such as from about 0.2 to about 6.0% by weight and in an example from about 0.5 to about 4.5% by weight.

In a further suitable embodiment, a process as contemplated herein is exemplified in that the agent (b) contains—based on the total weight of the agent (b)—one or more direct dyes in a total amount of from about 0.01 to about 10.0% by weight, in an exemplary embodiment from about 0.1 to about 8.0% by weight, such as from about 0.2 to about 6.0% by weight and in an example from about 0.5 to about 4.5% by weight.

### Other Ingredients

The agents (a) and (b) used in the process described above may further contain one or more additional optional ingredients.

The products may also contain one or more surfactants. The term surfactants refer to surface-active substances. A distinction is made between anionic surfactants including a hydrophobic residue and a negatively charged hydrophilic head group, amphoteric surfactants, which carry both a negative and a compensating positive charge, cationic surfactants, which in addition to a hydrophobic residue have a positively charged hydrophilic group, and non-ionic surfactants, which have no charges but strong dipole moments and are strongly hydrated in aqueous solution.

Zwitterionic surfactants are those surface-active compounds which carry at least one quaternary ammonium group and at least one —COO⁽⁻⁾— or —SO₃⁽⁻⁾ group in the molecule. Particularly suitable zwitterionic surfactants are the so-called betaines such as the N-alkyl-N,N-dimethylammonium-glycinate, for example the cocoalkyl-dimethylammoniumglycinate, N-acylaminopropyl-N,N-dimethylammoniumglycinate, for example, cocoacylaminopropyl dimethyl ammonium glycinate, and 2-alkyl-3-carboxymethyl-3-hydroxyethyl imidazolines each having 8 to 18 C atoms in the alkyl or acyl group, and cocoacylaminoethyl hydroxyethyl carboxymethyl glycinate. A suitable zwitterionic surfactant is the fatty acid amide derivative known under the INCI name cocamidopropyl betaine.

Ampholytic surfactants are surface-active compounds which, apart from a C₈-C₂₄ alkyl or acyl group, contain at least one free amino group and at least one —COOH— or —SO₃H group in the molecule and can form internal salts. Examples of suitable ampholytic surfactants are N-alkylglycines, N-alkylpropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids and alkylaminoacetic acids each with about 8 to 24 C atoms in the alkyl group. Typical examples of amphoteric or zwitterionic surfactants are alkylbetaines, alkylamidobetaines, amino propionates, aminoglycinate, imidazoliniumbetaines and sulfobetaines.

Particularly suitable ampholytic surfactants are N-cocoalkylaminopropionate, cocoacylaminoethylaminopropionate and C₁₂-C₁₈ acylsarcosine.

The products may also additionally contain at least one non-ionic surfactant. Suitable non-ionic surfactants are alkyl polyglycosides as well as alkylene oxide addition products to fatty alcohols and fatty acids with 2 to 30 mol ethylene oxide per mol fatty alcohol or fatty acid. Preparations with good properties are also obtained if they contain as non-ionic surfactants fatty acid esters of ethoxylated glycerol reacted with at least 2 mol ethylene oxide. The non-ionic surfactants are used in a total quantity of about 0.1 to about 45% by weight, in an exemplary embodiment from about 1 to about 30% by weight and in an example from about 1 to about 15% by weight—based on the total weight of the respective agent.

In addition, the products may also contain at least one cationic surfactant. Cationic surfactants are surfactants, i.e., surface-active compounds, each with one or more positive charges. Cationic surfactants contain only positive charges. Usually, these surfactants are composed of a hydrophobic part and a hydrophilic head group, the hydrophobic part usually including a hydrocarbon backbone (e.g., including one or two linear or branched alkyl chains) and the positive charge(s) being in the hydrophilic head group. Examples of cationic surfactants are
quaternary ammonium compounds which, as hydrophobic radicals, may carry one or two alkyl chains with a chain length of 8 to 28 C atoms,
quaternary phosphonium salts substituted with one or more alkyl chains with a chain length of 8 to 28 C atoms or
tertiary sulfonium salts.

Furthermore, the cationic charge can also be part of a heterocyclic ring (e.g., an imidazolium ring or a pyridinium ring) in the form of an onium structure. In addition to the functional unit carrying the cationic charge, the cationic surfactant may also contain other uncharged functional groups, as is the case for example with esterquats. The cationic surfactants are used in a total quantity of about 0.1 to about 45 wt. %, in an exemplary embodiment from about 1 to about 30 wt. % and in an example from about 1 to about 15 wt. %-based on the total weight of the respective agent.

Furthermore, the means as contemplated herein may also contain at least one anionic surfactant. Anionic surfactants are surface-active agents with exclusively anionic charges (neutralized by a corresponding counter cation). Examples of anionic surfactants are fatty acids, alkyl sulphates, alkyl ether sulphates and ether carboxylic acids with 12 to 20 C atoms in the alkyl group and up to 16 glycol ether groups in the molecule.

The anionic surfactants are used in a total quantity of about 0.1 to about 45 wt. %, in an exemplary embodiment from about 1 to about 30 wt. % and in an example from about 1 to about 15 wt. %—based on the total weight of the respective agent.

They may also contain other active substances, auxiliaries and additives, such as solvents, fatty components such as C8-C30 fatty alcohols, C₈-C₃₀ fatty acid triglycerides, C₈-C₃₀ fatty acid monoglycerides, C₈-C₃₀ fatty acid diglycerides and/or hydrocarbons; structural agents such as glucose, maleic acid and lactic acid; hair conditioning compounds such as phospholipids, for example lecithin and cephalins; perfume oils, dimethylisosorbide and cyclodextrins; fiber structure-improving active substances, in particular mono-, di- and oligosaccharides such as glucose, galactose, fructose, fructose and lactose; dyes for coloring the composition; anti-dandruff active substances such as Piroctone Olamine, Zinc Omadine and Climbazol; amino acids and oligopeptides; protein hydrolysates on animal and/or vegetable basis, as well as in the form of their fatty acid condensation products or optionally anionic or cationically modified derivatives; vegetable oils; sunscreens and UV-blockers; active ingredients such as panthenol, pantothenic acid, pantolactone, allantoin, pyrrolidinonecarboxylic acids and their salts, and bisabolol; polyphenols, in particular hydroxycinnamic acids, 6,7-dihydroxycumarine, hydroxybenzoic acids, catechine, tannine, leukoanthocyanidine, anthocyanidine, flavanone, flavone and flavonols; ceramides or pseudoceramides; vitamins, provitamins and vitamin precursors; plant extracts; fats and waxes such as fatty alcohols, beeswax, montan wax and paraffins; swelling and penetrating substances such as glycerol, propylene glycol monoethyl ether, carbonates, hydrogen carbonates, guanidines, ureas and primary, secondary and tertiary phosphates; opacifiers such as latex, styrene/PVP and styrene/acrylamide copolymers; pearlescent agents such as ethylene glycol mono- and distearate and PEG-3 distearate; and blowing agents such as propane-butane mixtures, N₂O, dimethyl ether, CO₂ and air.

The selection of these other substances will be made by the specialist according to the desired properties of the agents. About other optional components and the quantities of these components used, explicit reference is made to the relevant manuals known to the specialist. The additional active ingredients and auxiliary substances are in an exemplary embodiment used in the preparations as contemplated herein in quantities of about 0.0001 to about 25 wt. % each, about 0.0005 to about 15 wt. %, based on the total weight of the respective agent.

### Process for Dyeing Keratin Materials

In the procedure as contemplated herein, agents (a) and (b) are applied to the keratinous materials, to human hair. Thus, agents (a) and (b) are the ready-to-use agents. The agents (a) and (b) are different.

In principle, agents (a) and (b) can be applied simultaneously or successively, whereby successive application is suitable.

The best results were obtained when agent (a) was first applied to the keratin materials in a first step and then agent (b) was applied in a subsequent step.

Quite particularly suitable, therefore, is a process for treating keratinous material, for coloring keratinous material, in particular human hair, comprising the following steps in the order indicated:
in a first step, applying a water-containing agent (a) to the keratinous material, the agent (a) having and containing a pH of at least 9.6:
(a1) at least one organic silicon compound selected from the group including silanes having one, two or three silicon atoms, and
(a2) at least one alkalizing agent selected from the group including ammonia, alkanolamines and basic amino acids, and
in a second step, applying an agent (b) to the keratinous material, the agent comprising (b):
(b1) at least one film-forming polymer.

Quite particularly suitable, therefore, is a process for treating keratinous material, for coloring keratinous material, in particular human hair, comprising the following steps in the order indicated:
in a first step, applying a water-containing agent (a) to the keratinous material, the agent (a) having and containing a pH of at least 9.6:
(a1) at least one organic silicon compound selected from the group including silanes having one, two or three silicon atoms, and
(a2) at least one alkalizing agent selected from the group including ammonia, alkanolamines and basic amino acids, and
in a second step, applying an agent (b) to the keratinous material, the agent comprising (b):
(b1) at least one film-forming polymer,
   exemplified in that the agent (a) and/or the agent (b) contains at least one colorant compound selected from the group including pigments and/or direct dyes.

The agents (a) and (b) are particularly in an exemplary embodiment applied within one and the same dyeing process, which means that there is a period of a maximum of several hours between the application of agents (a) and (b).

In a further suitable embodiment, a method as contemplated herein is exemplified in that first the agent (a) is applied, and then the agent (b) is applied, the time between the application of the agents (a) and (b) being at most 24 hours, in an exemplary embodiment at most 12 hours and such as at most 6 hours.

Characteristic of the agent (a) is its content of water, at least one reactive organic silicon compound (a1) and at least one alkalizing agent (a2). The pH value of the agent (a) is in the alkaline to strongly alkaline range with a value of at least 9.6.

The reactive organic silicon compound(s) (a1) undergoes an oligomerization or polymerization reaction and thus functionalizes the hair surface as soon as it meets it. In this way, a first, film is formed. The addition of the alkalizing agent (a2) optimally adapts the oligomerization rate to the hair treatment process. In the second step of the process, a second, polymer-containing agent (b) is now applied to the hair. During the application of agent (b), the film-forming polymers interact with the silane film and are thus bound to the keratin materials. Here, the technical application properties of the resulting dyeing can be further improved by selecting the optimum process conditions.

In the context of a further form of execution, a procedure comprising the following steps in the order indicated is particularly suitable

(1) Application of agent (a) on the keratinous material,
(2) Allow the agent (a) to act for a period of 10 seconds to 10 minutes, in an exemplary embodiment from 10 seconds to 5 minutes,
(3) if necessary, rinse the keratinous material with water,
(4) Application of agent (b) on the keratinous material,
(5) Allow the agent (b) to act for a period of 30 seconds to 30 minutes, in an exemplary embodiment from 30 seconds to 10 minutes, and
(6) Rinse the keratinous material with water.

The rinsing of the keratinous material with water in steps (3) and (6) of the process is understood, as contemplated herein, to mean that only water is used for the rinsing process, without any other agents other than agents (a) and (b).

In a first step (1), agent (a) is applied to the keratin materials, especially human hair.

After application, the agent (a) can act on the keratin materials. In this context, application times from about 10 seconds to about 10 minutes, in an exemplary embodiment from about 20 seconds to about 5 minutes and for example from about 30 seconds to about 2 minutes on the hair have proven to be particularly beneficial.

In a suitable embodiment of the method as contemplated herein, the agent (a) can now be rinsed from the keratin materials before the agent (b) is applied to the hair in the subsequent step.

Dyeing's with also good wash fastness were obtained when agent (b) was applied to the keratin materials which were still exposed to agent (a).

In step (4), agent (b) is now applied to the keratin materials. After application, let the agent (b) act on the hair.

The process as contemplated herein allows the production of dyeing's with particularly good intensity and wash fastness even with a short exposure time of agent (b). Application times from about 10 seconds to about 10 minutes, in an exemplary embodiment from about 20 seconds to about 5 minutes and in an example from about 30 seconds to about 3 minutes on the hair have proven to be particularly beneficial.

In step (6), agent (b) (and any remaining agent (a)) is rinsed out of the keratin material with water.

In the context of a further form of execution, a procedure comprising the following steps in the order indicated is particularly suitable

(1) Application of agent (a) on the keratinous material,
(2) Allow the agent (a) to act for a period of about 10 seconds to about 10 minutes, in an exemplary embodiment from about from 10 seconds to about 5 minutes,
(3) if necessary, rinse the keratinous material with water,
(4) Application of agent (b) on the keratinous material,
(5) Allow the agent (b) to act for a period of about 30 seconds to about 30 minutes, in an exemplary embodiment from about 30 seconds to about 10 minutes, and
(6) Rinse the keratinous material with water.

In this embodiment, the sequence of steps (1) to (6) in an exemplary embodiment takes place within 24 hours.

The agent (a) contains with the organic silicon compound(s) a class of highly reactive compounds which can undergo hydrolysis and/or oligomerization and/or polymerization in the presence of water/alkalizing agents as described above. Due to their high reactivity, these organic silicon compounds form a film on the keratin material.

To avoid premature oligomerization or polymerization, it is of considerable advantage to the user to prepare the ready-to-use agent (a) only shortly before application.

In yet another embodiment, suitable is a method comprising the following steps in the order indicated.

(1) preparing an agent (a) by mixing a first agent (a′) and a second agent (a″), wherein
the first agent (a′) includes at least one organic silicon compound (a1) from the group of silanes having one, two or three silicon atoms, and
the second agent (a″) has a pH of at least 9.6 and includes water and at least one alkalizing agent (a2) selected from the group including ammonia, alkanolamines and basic amino acids,
   (2) Application of agent (a) on the keratinous material,
   (3) Allow the agent (a) to act for a period of about 10 seconds to about 10 minutes, in an exemplary embodiment from about 10 seconds to about 5 minutes,
   (4) if necessary, rinse the keratinous material with water,
   (4) Application of agent (b) on the keratinous material,
   (5) Allowing the agent (b) to act for a period of about 30 seconds to about 30 minutes, in an exemplary embodiment from about 30 seconds to about 10 minutes,
   (6) if necessary, rinse the keratinous material with water.

To be able to provide a formulation that is as stable as possible in storage, the agent (a′) itself is in an exemplary embodiment formulated to be low in water or water-free.

In a suitable embodiment, a multicomponent packaging unit (kit-of-parts) as contemplated herein is exemplified in that the agent (a′)—based on the total weight of the agent (a′)—contains a water content of from about 0.001 to about 10.0% by weight, in an exemplary embodiment from about 0.5 to about 9.0% by weight, such as from about 1.0 to about 8.0% by weight and in an embodiment from about 1.5 to about 7.0% by weight.

The agent (a″) contains water. In a suitable embodiment, a multicomponent packaging unit (kit-of-parts) as contemplated herein is exemplified in that the agent (a″)—based on the total weight of the agent (a2)—has a water content of from about 15 to about 100% by weight, in an exemplary embodiment from about 35 to about 100% by weight, such as from about 55 to about 100% by weight, such as from about 65 to about 100% by weight and in an embodiment from about 75 to about 100% by weight.

Within this embodiment, the ready-to-use agent (a) is now prepared by mixing agents (a′) and (a″).

The agent (a″) has a pH of at least 9.6, which ensures that the ready-to-use agent (a) prepared by mixing agents (a′) and (a″) also has a pH of at least 9.6.

For example, the user may first stir or shake the agent (a′) containing the organic silicon compound(s) (a1) with the aqueous alkaline agent (a″). The user can now apply this mixture of (a′) and (a″) to the keratin materials—either immediately after its preparation or after a short reaction time of 10 seconds to 20 minutes. Afterwards, the user can apply agent (b) as described above.

To hair coloring, the coloring compound from the group of pigments or direct dyes can now be incorporated into the agent (a′), into the agent (a″) or also into the agent (b).

In a further suitable embodiment, a process as contemplated herein is exemplified in that the agent (a′), the agent (a″) and/or the agent (b) contains at least one colorant compound from the group including pigments and/or direct dyes.

In yet another embodiment, suitable is a method comprising the following steps in the order indicated.

(1) preparing an agent (a) by mixing a first agent (a′) and a second agent (a″), wherein
the first agent (a′) includes at least one organic silicon compound (a1) from the group of silanes having one, two or three silicon atoms and furthermore at least one colorant compound from the group of pigments and/or direct dyes (a3), and
the second agent (a″) has a pH of at least 9.6 and includes water and at least one alkalizing agent (a2) selected from the group including ammonia, alkanolamines and basic amino acids,
   (2) Application of agent (a) on the keratinous material,
   (3) Allow the agent (a) to act for a period of 10 seconds to 10 minutes, in an exemplary embodiment from 10 seconds to 5 minutes,
   (4) if necessary, rinse the keratinous material with water,
   (4) Application of agent (b) on the keratinous material,
   (5) Allowing the agent (b) to act for a period of about 30 seconds to about 30 minutes, in an exemplary embodiment from about 30 seconds to about 10 minutes,
   (6) if necessary, rinse the keratinous material with water.

In yet another embodiment, suitable is a method comprising the following steps in the order indicated.

(1) preparing an agent (a) by mixing a first agent (a′) and a second agent (a″), wherein
the first agent (a′) includes at least one organic silicon compound (a1) from the group of silanes having one, two or three silicon atoms, and
the second agent (a″) has a pH of at least 9.6 and includes water and at least one alkalizing agent (a2) selected from the group including ammonia, alkanolamines and basic amino acids and furthermore at least one colorant compound selected from the group including pigments and/or direct dyes (a3),
   (2) Application of agent (a) on the keratinous material,
   (3) Allow the agent (a) to act for a period of about 10 seconds to about 10 minutes, in an exemplary embodiment from about 10 seconds to about 5 minutes,
   (4) if necessary, rinse the keratinous material with water,
   (4) Application of agent (b) on the keratinous material,
   (5) Allowing the agent (b) to act for a period of about 30 seconds to about 30 minutes, in an exemplary embodiment from about 30 seconds to about 10 minutes,
   (6) if necessary, rinse the keratinous material with water.

In yet another embodiment, suitable is a method comprising the following steps in the order indicated.

(1) preparing an agent (a) by mixing a first agent (a′) and a second agent (a″), wherein
the first agent (a′) includes at least one organic silicon compound (a1) from the group of silanes having one, two or three silicon atoms, and
the second agent (a″) has a pH of at least 9.6 and includes water and at least one alkalizing agent (a2) selected from the group including ammonia, alkanolamines and basic amino acids,
   (2) Application of agent (a) on the keratinous material,
   (3) Allow the agent (a) to act for a period of about 10 seconds to about 10 minutes, in an exemplary embodiment from about 10 seconds to about 5 minutes,
   (4) if necessary, rinse the keratinous material with water,
   (4) Application of agent (b) to the keratinous material, wherein.
the composition (b) includes at least one film-forming polymer (b1) and furthermore at least one colorant compound (b2) selected from the group including pigments and/or direct dyes,
   (5) Allowing the agent (b) to act for a period of about 30 seconds to about 30 minutes, in an exemplary embodiment from about 30 seconds to about 10 minutes,
   (6) if necessary, rinse the keratinous material with water.

Furthermore, it is also possible to make up the colorant compound in a separate, third means (a″).

Suitable in the context of this further embodiment is a method comprising the following steps in the order indicated.

(1) preparing an agent (a) by mixing a first agent (a′) and a second agent (a″) and a third agent (a″), wherein
the first agent (a′) includes at least one organic silicon compound (a1) from the group of silanes having one, two or three silicon atoms, and
the second agent (a″) has a pH of at least 9.6 and includes water and at least one alkalizing agent (a2) selected from the group including ammonia, alkanolamines and basic amino acids, and
the third agent (a′″) includes at least one colorant compound (a3) from the group including pigments and/or direct dyes,
   (2) Application of agent (a) on the keratinous material,
   (3) Allow the agent (a) to act for a period of about 10 seconds to about 10 minutes, in an exemplary embodiment from about 10 seconds to about 5 minutes,
   (4) if necessary, rinse the keratinous material with water,
   (4) Application of agent (b) on the keratinous material,
   (5) Allowing the agent (b) to act for a period of about 30 seconds to about 30 minutes, in an exemplary embodiment from about 30 seconds to about 10 minutes,
   (6) if necessary, rinse the keratinous material with water.

In the previously described embodiments, the colorant compounds were named from the group including pigments and direct dyes depending on the medium in which they were made up. When the colorant compounds were made up in an agent (a) (or (a′), (a″) or (a′″)), they were designated (a3). When the colorant compounds were made up in an agent (b), they were designated (b2).

### Multi-Component Packaging Unit (Kit-of-Parts)

To increase user comfort, the user is in an exemplary embodiment provided with all required resources in the form of a multi-component packaging unit (kit-of-parts).

Thus, a second object of the present disclosure is a multi-component packaging unit (kit-of-parts) for treating keratinous material, comprising separately assembled:
a first container with an agent (a′), wherein the agent (a′) contains at least one organic silicon compound (a1) from the group of silanes with one, two or three silicon atoms, and
a second container comprising an agent (a′), said agent (a″) having a pH of at least 9.6 and comprising water and at least one alkalizing agent (a2) selected from the group including ammonia, alkanolamines and basic amino acids, and
a third container comprising an agent (b), wherein the agent (b) includes at least one film-forming polymer (b1),
   wherein the components (a1), (a2) and (b1) were disclosed in detail in the description of the first subject matter of the invention.

The organic silicon compounds (a1) from the group of silanes with one, two or three silicon atoms contained in agent (a) of the kit correspond to the organic silicon compounds that were also used in agent (a) of the previously described process.

The alkalizing agents (a2) from the group including ammonia, alkanolamines and basic amino acids contained in the agent (a″) of the kit correspond to the alkalizing agents also used in agent (a) of the previously described process.

The film-forming polymers (b1) contained in agent (b) of the kit correspond to the film-forming polymers that were also used in agent (b) of the previously described process.

The multi-component packaging unit of the second invention as contemplated herein is also in an exemplary embodiment used for coloring the keratin material, in particular human hair.

For this reason, it is further particularly suitable if the agent (a′), the agent (a″) and/or the agent (b) contains at least one colorant compound from the group of pigments and/or direct dyes.

In a further suitable embodiment, the multicomponent packaging unit (kit-of-parts) as contemplated herein, exemplified in that the agent (a′), the agent (a″) and/or the agent (b) contains at least one colorant compound from the group including pigments and/or direct dyes.

The colorant compounds that can be used in means (a′), (a″) and (b) of the kit again correspond to the colorant compounds disclosed in detail in the description of the first subject matter of the invention.

In this context, it is again possible to make up the color-imparting compound in a separate, third means (a″). In the context of this further embodiment, the multi-component packaging unit (kit-of-parts) therefore also includes a third agent (a″), which contains at least one color-imparting compound.

In an exemplary embodiment, therefore, a multi-component packaging unit (kit-of-parts) for treating keratinous material, comprising separately packaged:
a first container with an agent (a′), wherein the agent (a′) contains at least one organic silicon compound (a1) from the group of silanes with one, two or three silicon atoms, and
a second container comprising an agent (a″), said agent (a″) having a pH value of at least 9.6 and comprising water and at least one alkalizing agent (a2) selected from the group including ammonia, alkanolamines and basic amino acids, and
a third container with an agent (a″), wherein the agent (a″) contains at least one colorant compound (a3) from the group of pigments and/or direct dyes, and
a fourth container comprising an agent (b), the agent (b) comprising at least one film-forming polymer (b1),
   wherein the components (a1), (a2), (a3) and (b1) are defined in any one of claims 1 to 19.

With respect to the other suitable embodiments of the multi-component packaging unit as contemplated herein, the same applies mutatis mutandis to the procedure as contemplated herein.

### EXAMPLES

### Example 1

The following formulations have been produced (unless otherwise indicated, all figures are in % by weight)

Agent (a′)

| Agent (a′) | in wt.. % |
|---|---|
| (3-Aminopropyl)triethoxysilane (a1) | 20.0 |
| Methyltrimethoxysilane (a1) | 60.0 |
| Phthalocyanine blue pigment CI 74160 (a3) | 5.0 |
| Coco-Glucoside | 5.0 |
| Water | ad 100 |

Agent (a″)

| Agent (a″) | in wt.. % |
|---|---|
| Ammonia (a2) | ad pH 10.0 |
| Hydroxyethyl cellulose | 1.0 |
| Water | ad 100 |

The ready-to-use agent (a) was prepared by mixing 5.0 g of agent (a′) and 20.0 g of agent (a″). The pH value of the agent (a) was adjusted to a value of 10.5 by further addition of ammonia or lactic acid. Then the agent (a) could stand for about 5 minutes.

Agent (b)

| Agent (b) | in wt.. % |
|---|---|
| Ethylene/Sodium Acrylate Copolymer (b1) | 40.0 |
| 25% solution | |
| Water | ad 100 |

The agent (a) was massaged into one strand of hair at a time (Kerling, Euronatural hair white), and left to act for 1 minute. The agent (a) was then rinsed with water. Subsequently, agent (b) was applied to the hair strand, left to act for 1 minute and then also rinsed with water.

An intense blue coloration with good wash fastness was obtained.

### Example 2

The following formulations have been produced (unless otherwise indicated, all figures are in % by weight)

Agent (a′)

| Agent (a′) | in wt.. % |
|---|---|
| (3-Aminopropyl)triethoxysilane (a1) | 20.0 |
| Methyltrimethoxysilane (a1) | 70.0 |
| Water | ad 100 |

Agent (a″)

| Agent (a″) | in wt.. % |
|---|---|
| Ammonia (a2) | ad pH 10.0 |
| Phthalocyanine blue pigment CI 74160 (a3) | 5.0 |
| Coco-Glucoside | 5.0 |
| Hydroxyethyl cellulose | 1.0 |
| Water | ad 100 |

The ready-to-use agent (a) was prepared by mixing 5.0 g of agent (a′) and 20.0 g of agent (a″). The pH value of the agent (a) was adjusted to a value of 10.5 by further addition of ammonia or lactic acid. Then the agent (a) could stand for about 5 minutes.

Agent (b)

| Agent (b) | in wt.. % |
|---|---|
| Ethylene/Sodium Acrylate Copolymer (b1) | 40.0 |
| 25% solution | |
| Water | ad 100 |

The agent (a) was massaged into one strand of hair at a time (Kerling, Euronatural hair white), and left to act for 1 minute. The agent (a) was then rinsed with water. Subsequently, agent (b) was applied to the hair strand, left to act for 1 minute and then also rinsed with water.

An intense blue coloration with good wash fastness was obtained.

### Example 3

The following formulations have been produced (unless otherwise indicated, all figures are in % by weight)

Agent (a′)

| Agent (a′) | in wt.. % |
|---|---|
| (3-Aminopropyl)triethoxysilane (a1) | 20.0 |
| Methyltrimethoxysilane (a1) | 70.0 |
| Water | ad 100 |

Agent (a″)

| Agent (a″) | in wt.. % |
|---|---|
| Ammonia (a2) | ad pH 10.0 |
| Hydroxyethyl cellulose | 1.0 |
| Water | ad 100 |

The ready-to-use agent (a) was prepared by mixing 5.0 g of agent (a′) and 20.0 g of agent (a″). The pH value of the agent (a) was adjusted to a value of 10.5 by further addition of ammonia or lactic acid. Then the agent (a) could stand for about 5 minutes.

Agent (b)

| Agent (b) | in wt.. % |
|---|---|
| Ethylene/Sodium Acrylate Copolymer (b1) | 40.0 |
| 25% solution | |
| Pigment Yellow 401, CI 11680 2-[(4- | 5.0 |
| Methyl-2-nitro)azo]3-oxo-N- | |
| phenylbutyramid (b2) | |
| Water | ad 100 |

The agent (a) was massaged into one strand of hair at a time (Kerling, Euronatural hair white), and left to act for 1 minute. The agent (a) was then rinsed with water. Subsequently, agent (b) was applied to the hair strand, left to act for 1 minute and then also rinsed with water.

An intense yellow coloration with good wash fastness was obtained.

### Example 4

The following formulations have been produced (unless otherwise indicated, all figures are in % by weight)

Agent (a′)

| Agent (a′) | in wt.. % |
|---|---|
| (3-Aminopropyl)triethoxysilane (a1) | 20.0 |
| Methyltrimethoxysilane (a1) | 70.0 |
| Water | ad 100 |

Agent (a″)

| Agent (a″) | in wt.. % |
|---|---|
| Ammonia (a2) | ad pH 10.5 |
| Hydroxyethyl cellulose | 1.0 |
| Water | ad 100 |

Agent (a″′)

| Agent (a″′) | in wt.. % |
|---|---|
| Pigment permanent red R CI 12085 1-[(2- | 60.0 |
| chloro-4-nitrophenyl)azo]-2-naphthol (a3) | |
| PEG-12 Dimethicone | 40.0 |

The ready-to-use agent (a) was prepared by mixing 5.0 g of agent (a′) and 20.0 g of agent (a″) and 5.0 g of agent (a″). The pH value of the agent (a) was adjusted to a value of 10.5 by further addition of ammonia or lactic acid. Then the agent (a) could stand for about 5 minutes.

Agent (b)

| Agent (b) | in wt.. % |
|---|---|
| Ethylene/Sodium Acrylate Copolymer (b1) | 40.0 |
| 25% solution | |
| Water | ad 100 |

The agent (a) was massaged into one strand of hair at a time (Kerling, Euronatural hair white), and left to act for 1 minute. The agent (a) was then rinsed with water. Subsequently, agent (b) was applied to the hair strand, left to act for 1 minute and then also rinsed with water.

An intense red coloration with good wash fastness was obtained.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A process for treating keratinous material, comprising the following steps:
applying a water-containing agent (a) to the keratinous material, wherein the agent (a) comprises a pH of at least about 9.6:
(a1) at least one organic silicon compound selected from the group consisting of silanes having one, two or three silicon atoms, and
(a2) at least one alkalizing agent selected from the group consisting of ammonia, alkanolamines and basic amino acids, and
applying an agent (b) to the keratinous material, wherein the agent (b) comprises:
(b1) at least one film-forming polymer.

2. The process according to claim 1, wherein the agent (a) has a pH of from about 9.7 to about 11.5.

3. The process according to claim 1, wherein the agent (a) comprises at least one organic silicon compound (a1) of the formula (I) and/or (II)
R₁R₂N-L-Si(OR₃)ₐ(R₄)_{b} (I),
where
R₁, R₂ independently represent a hydrogen atom or a C₁-C₆ alkyl group,
L is a linear or branched divalent C₁-C₂₀ alkylene group,
R₁, R₄ independently of one another represent a C₁-C₆ alkyl group,
a, stands for an integer from 1 to 3, and
b stands for the integer 3-a, and
wherein in the organic silicon compound of formula (II)
(R₅O)_{c}(R₆)_{d}Si-(A)ₑ-[NR₇-(A′)]_{f}—[O-(A″)]_{g}—[NR₈-(A′″)]ₕ—Si(R₆′)_{d′}(OR₅′)_{c′} (II),
R5, R5′, R5″, R6, R6′ and R6″ independently represent a C₁-C₆ alkyl group,
A, A′, A″, A′″ and A″″ independently represent a linear or branched divalent C₁-C₂₀ alkylene group,
R₇ and R₈ independently represent a hydrogen atom, a C₁-C₆ alkyl group, a hydroxy C₁-C₆ alkyl group, a C₂-C₆ alkenyl group, an amino C₁-C₆ alkyl group or a group of formula (III)
-(A″″)-Si(R₆″)_{d}″(OR₅″)_{c}″ (III),
c, stands for an integer from 1 to 3,
d stands for the integer 3-c,
c′ stands for an integer from 1 to 3,
d′ stands for the integer 3-c′,
c″ stands for an integer from 1 to 3,
d″ stands for the integer 3-c″,
e stands for 0 or 1,
f stands for 0 or 1,
g stands for 0 or 1,
h stands for 0 or 1,
provided that at least one of e, f, g, and h is different from 0.

4. The method according to claim 1, wherein the agent (a) comprises at least one organic silicon compound (a10 of formula (I),
R₁R₂N-L-Si(OR₃)ₐ(R₄)_{b} (I),
where
R₁, R₂ both represent a hydrogen atom, and
L represents a linear, divalent C₁-C₆-alkylene group,
R₃, R₄ independently represent a methyl group or an ethyl group, and
a stands for the number 3 and
b stands for the number 0.

5. The agent according to claim 1, wherein the agent (a) comprises at least one organic silicon compound (a1) of formula (I) selected from the group of
(3-Aminopropyl)trimethoxysilane
(3-Aminopropyl)triethoxysilan
(2-Aminoethyl)trimethoxysilane
(2-Aminoethyl)triethoxysilan
(3-Dimethylaminopropyl)trimethoxysilane
(3-Dimethylaminopropyl)triethoxysilan
(2-dimethylaminoethyl)trimethoxysilane and
(2-Dimethylaminoethyl)triethoxysilan.

6. The process according to claim 1, wherein the agent (a) comprises at least one organic silicon compound (a1) of formula (II).
(R₅O)_{c}(R₆)_{d}Si-(A)ₑ-[NR₇-(A′)]_{f}—[O-(A″)]_{g}—[NR₈-(A′″)]ₕ—Si(R₆′)_{d′}(OR₅′)_{c′} (II),
where
e and f both stand for the number 1,
g and h both stand for the number 0,
A and A′ independently represent a linear, divalent C₁-C₆ alkylene group and
R7 represents a hydrogen atom, a methyl group, a 2-hydroxyethyl group, a 2-alkenyl group, a 2-aminoethyl group or a group of formula (III).

7. The process according to claim 1, wherein the agent (a) comprises at least one organic silicon compound (a1) of formula (II) selected from the group of
3-(trimethoxysilyl)-N-[3-(trimethoxysilyl)propyl]-1-propanamine
3-(Triethoxysilyl)-N-[3-(triethoxysilyl)propyl]-1-propanamine
N-methyl-3-(trimethoxysilyl)-N-[3-(trimethoxysilyl)propyl]-1-propanamine
N-Methyl-3-(triethoxysilyl)-N-[3-(triethoxysilyl)propyl]-1-propanamine
2-[Bis[3-(trimethoxysilyl)propyl]amino]-ethanol
2-[bis[3-(triethoxysilyl)propyl]amino]ethanol
3-(Trimethoxysilyl)-N,N-bis[3-(trimethoxysilyl)propyl]-1-propanamine
3-(Triethoxysilyl)-N,N-bis[3-(triethoxysilyl)propyl]-1-propanamine
N1,N1-bis[3-(trimethoxysilyl)propyl]-1,2-ethanediamine,
N1,N1-bis[3-(triethoxysilyl)propyl]-1,2-ethanediamine,
N,N-bis[3-(trimethoxysilyl)propyl]-2-propen-1-amine and/or
N,N-bis[3-(triethoxysilyl)propyl]-2-propen-1-amine.

8. The process according to claim 1, wherein the agent (a) comprises at least one organic silicon compound (a1) of formula (IV).
R₉Si(OR₁₀)ₖ(R₁₁)ₘ (IV),
where
R₉ represents a C₁-C₁₂ alkyl group,
R₁₀ represents a hydrogen atom or a C₁-C₆ alkyl group,
R₁₁ represents a C₁-C₆ alkyl group
k is an integer from 1 to 3, and
m stands for the integer 3-k.

9. The process according to claim 1, wherein the agent (a) comprises at least one organic silicon compound (a1) of formula (IV) selected from the group of
Methyltrimethoxysilane
Methyltriethoxysilane
Ethyltrimethoxysilane
Ethyltriethoxysilane
Octyltrimethoxysilane
Octyltriethoxysilane
Dodecyltrimethoxysilane and/or
Dodecyltriethoxysilane.

10. Process according to claim 1, wherein the agent (a) comprises at least two structurally different organic silicon compounds (a1).

11. Process according to claim 1, wherein the agent (a)—based on the total weight of agent (a)—comprises:
from about 0.5 to about 5.0 wt. % of at least one first organic silicone compound (a1) selected from the group of (3-aminopropyl)trimethoxysilane, (3-aminopropyl)triethoxysilane, (2-aminoethyl)trimethoxysilane, (2-aminoethyl)triethoxysilane, (3-dimethylaminopropyl)trimethoxysilane, (3-dimethylaminopropyl)triethoxysilane (2-dimethylaminoethyl)trimethoxysilane and (2-dimethylaminoethyl)triethoxysilane, and
from about 3.2 to about 10.0% by weight of at least one second organic silicon compound (a1) selected from the group of methyltrimethoxysilane, methyltriethoxysilane, ethyltrimethoxysilane, ethyltriethoxysilane, octyltrimethoxysilane, octyltriethoxysilane, dodecyltrimethoxysilane and dodecyltriethoxysilane.

12. The method according to claim 1, wherein the agent (a) comprises ammonia as alkalizing agent (a2).

13. Process according to claim 1, wherein the agent (a) comprises at least one alkalizing agent (a2) from the group of alkanolamines from 2 aminoethan-1-ol (monoethanolamine), 3-aminopropan-1-ol, 4-aminobutan-1-ol, 5-aminopentan-1-ol, 1-aminopropan-2-ol, 1-aminobutan-2-ol, 1-aminopentan-2-ol, 1-aminopentan-3-ol, 1-aminopentan-4-ol, 3-amino-2-methylpropan-1-ol, 1-amino-2-methylpropan-2-ol, 3-aminopropane-1,2-diol and 2-amino-2-methylpropane-1,3-diol.

14. Process according to claim 1, wherein the agent (a) comprises at least one alkalizing agent (a2) from the group of basic amino acids, from arginine, lysine, ornithine, and histidine.

15. A process according to claim 1, wherein the total amount of organic acids from the group consisting of citric acid, tartaric acid, malic acid, and lactic acid included in the agent (a) is below about 1.0% by weight.

16. The method according to claim 1, wherein the agent (b) is
at least one film-forming polymer (b1) selected from the group of homopolymers and copolymers of acrylic acid, homopolymers and copolymers of methacrylic acid, homopolymers and copolymers of acrylic acid esters, homopolymers and copolymers of methacrylic acid esters, homopolymers and copolymers of acrylic amides homopolymers and copolymers of methacrylic acid amides, homopolymers and copolymers of vinylpyrrolidone, homopolymers and copolymers of vinyl alcohol, homopolymers and copolymers of vinyl acetate, homopolymers and copolymers of ethylene, homopolymers and copolymers of propylene, homopolymers and copolymers of styrene, polyurethanes, polyesters and polyamides.

17. The process according to claim 1, wherein the agent (b) comprises at least one film-forming polymer (b1) comprising at least one structural unit of the formula (P-I) and at least one structural unit of the formula (P-II) 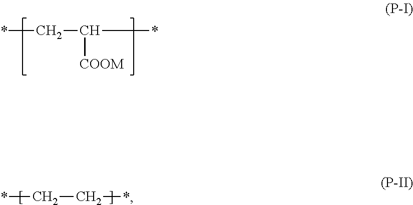
where
M is a hydrogen atom or ammonium (NH₄), sodium, potassium, ½ magnesium or ½ calcium.

18. Process according to claim 1, wherein the agent (b) comprises—based on the total weight of the agent (b)—one or more film-forming polymers (b1) in a total amount of from about 0.1 to about 18.0% by weight.

19. The process according to claim 1, wherein the agent (a) and/or the agent (b) comprises at least one colorant compound selected from the group of pigments and/or direct dyes.

20. Process according to claim 1, wherein the agent (a) and/or the agent (b) comprises at least one inorganic pigment selected from the group of colored metal oxides, metal hydroxides, metal oxide hydrates, silicates, metal sulfides, complex metal cyanides, metal sulfates, bronze pigments and/or from colored mica- or mica-based pigments coated with at least one metal oxide and/or a metal oxychloride.

21.-31. (canceled)
